SEQUENCE LISTING (103 sequences)

SEQ ID NO: 1 - protein, 39 aa, Human
MADEEKLPPGWEKRMSRSSGRVYYFNHITNASQWERPSG

SEQ ID NO: 2 - protein, 38 aa, Artificial Sequence
  WW Domain Peptide Library
MADEEKLPPGWXKXWSXXGRVXYXNXITXAXQWERPSG

SEQ ID NO: 3 - DNA, 114 bp, Artificial Sequence
  WW Domain Nucleic Acid Library
atggccgatgaagagaaactgccgccaggctggnnbaaannbtggagtvvmvvmggacgcgtcnnbtacnnbaatnnbatcactnnbgcgvvmcagtgggaacgaccatcgggc

SEQ ID NO: 4 - DNA, 52 bp, Artificial Sequence
  1StepRepFor Primer
ggcagcggttctagtctagcggccccaactgatcttcaccaaacgtattacc

SEQ ID NO: 5 - DNA, 17 bp, Artificial Sequence
  M13rev Primer
caggaaacagctatgac

SEQ ID NO: 6 - DNA, 22 bp, Artificial Sequence
  DigLigRev Primer
catgattacgccaagctcagaa

SEQ ID NO: 7 - DNA, 131 bp, Artificial Sequence
  131mer Primer
cggcggttagaacgcggctacaattaatacataaccccatccccctgttgacaattaatcatcggctcgtataatgtgtggaattgtgagcggataacaatttcacacaggaaacaggatctaccatggcc

SEQ ID NO: 8 - protein, 922 aa, Artificial Sequence
  VEGFR2-Fc Fusion Peptide
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 27 bp, Artificial Sequence
  R1RecFor Primer
gaacgcggctacaattaatacataacc

SEQ ID NO: 10 - DNA, 26 bp, Artificial Sequence
  NotRecRev Primer
tggtgaagatcagttgcggccgctag

SEQ ID NO: 11 - DNA, 26 bp, Artificial Sequence
  TAC6 Primer
ccccatccccctgttgacaattaatc

SEQ ID NO: 12 - protein, 38 aa, Artificial Sequence
  Mutated WW Domain Pin1 Peptide
MADEEKLPPGWEKRWSRSGRVYYFNHITNASQWERPSG

SEQ ID NO: 13 - DNA, 150 bp, Artificial Sequence
  WW Domain Nucleic Acid Library and Linker
ggaaacaggatctaccatggccgatgaagagaaactgccgccaggctggnnbaaannbtggagtvvmvvmggacgcgtcnnbtacnnbaatnnbatcactnnbgcgvvmcagtgggaacgaccatcgggcggcagcggttctagtctagc

SEQ ID NO: 14 - protein, 43 aa, Artificial Sequence
  WW Domain Peptide Library and Linker
MADEEKLPPGWXKXWSXXGRVXYXNXITXAXQWERPSGGSGSS

SEQ ID NO: 15 - protein, 38 aa, Artificial Sequence
  Mutant WW Domain Peptide
MADEEKLPPGWYKMWSSPGRVLYVNDITHAHRWERPSG

SEQ ID NO: 16 - protein, 38 aa, Artificial Sequence
  Mutant WW Domain Peptide
MADEEKLPPGWYKMWSSPGRVLYVNDITHAHQWERPSG

SEQ ID NO: 17 - protein, 38 aa, Artificial Sequence; identical to SEQ ID NO: 16
  Mutant WW Domain Peptide

SEQ ID NO: 18 - protein, 38 aa, Artificial Sequence
  Mutant WW Domain Peptide
MAVEEKLPPGWYKMWSSPGRVLYVNDITHAHQWERPSG

SEQ ID NO: 19 - protein, 38 aa, Artificial Sequence
  Mutant WW Domain Peptide
MADEEKLPPGWGKGWSPRGRVWYTNRTTVAAQWERPSG

SEQ ID NO: 20 - protein, 38 aa, Artificial Sequence
  Mutant WW Domain Peptide
MADEEKLPPGWCKFWSTAGRVIYINRITGAKQWERPSG

SEQ ID NO: 21 - protein, 118 aa, human
SSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRR

SEQ ID NO: 22 - DNA, 54 bp, Artificial Sequence
  NotRecRevWW1 Primer
agatcagttgcggccgcagagcttgctacggtgctgcccgatggtcgttcccac

SEQ ID NO: 23 - DNA, 29 bp, Artificial Sequence
  Tac6-9 Primer
cggctcgtataatgtgtggaattgtgagc

SEQ ID NO: 24 - DNA, 26 bp, Artificial Sequence
  NotRecRevWW2 Primer
agatcagttgcggccgcagagcttgc

SEQ ID NO: 25 - DNA, 110 bp, Artificial Sequence
  110-mer Primer
cggcggttagaacgcggctacaattaatacataaccccatccccctgttgacaattaatcatggctcgtataatgtgtggaattgtgagcggataacaatttcacacagg

SEQ ID NO: 26 - protein, 37 aa, Artificial Sequence
  Mutant WW Domain Peptide
MADEEKLPPGWAVWSPDGRLYYWNSVIVSTRWGRPSC

SEQ ID NO: 27 - protein, 37 aa, Artificial Sequence
  Mutant WW Domain Peptide
MADEEKLPPGWTVWSPNGRVYYWNSITVARQWERPSG

SEQ ID NO: 28 - protein, 38 aa, Artificial Sequence
  Mutant WW Domain Peptide
MADEEKLPPGWFKCWGRGGRVWYMNWITVAGQWERPSG

SEQ ID NO: 29 - protein, 38 aa, Artificial Sequence
  Mutant WW Domain Peptide
MADEEKLPPGWGKPWSARGRVTYINLITVAEQWERPSG

SEQ ID NO: 30 - protein, 36 aa, Artificial Sequence
  Cyclic WW Domain Peptide
DEEKLPPGWYKMWSSPGRVLYVNDITHAHRWERPEG

SEQ ID NO: 31 - protein, 32 aa, Artificial Sequence
  WW Domain Peptide Library
KLPPGWXKXWSXXXGRVXYXNXITXAXQWERP

SEQ ID NO: 32 - protein, 31 aa, Artificial Sequence
  WW Domain Peptide Library
KLPPGWXKXWSXXGRVXYXNXITXAXQWERP

SEQ ID NO: 33 - protein, 5 aa, Artificial Sequence
  Linker Peptide
GSGSS

SEQ ID NO: 34 - protein, 36 aa, Artificial Sequence
  Cyclic WW Domain Peptide
DEEKLPPGWYKMWSSPGRVLYVNDIKHAHRWERPEG

SEQ ID NO: 35 - protein, 39 aa, Pan troglodytes; identical to SEQ ID NO: 1

SEQ ID NO: 36 - protein, 39 aa, Canis familiaris; identical to SEQ ID NO: 1

SEQ ID NO: 37 - protein, 39 aa, Bos taurus; identical to SEQ ID NO: 1

SEQ ID NO: 38 - protein, 39 aa, Mus musculus; identical to SEQ ID NO: 1

SEQ ID NO: 39 - protein, 42 aa, Rattus norvegicus
GGKMADEEKLPSGWEKRMSRSSGRVYYFNHITNASQWERPSG

SEQ ID NO: 40 - protein, 39 aa, Drosophila melanogaster
MPDAEQLPDGWEKRTSRSTGMSYYLNMYTKESQWDQPTE

SEQ ID NO: 41 - protein, 40 aa, Anopheles gambiae
MSDGQETVPEGWEKRTSRSTGMTYYLNVYTKESQWDPPTA

SEQ ID NO: 42 - protein, 38 aa, Caenorhabditis elegans
MSDNSLPAGWEKRQSRSNDRVYYFNTATGRSQWERPDE

SEQ ID NO: 43 - protein, 38 aa, Schizosaccharomyces pombe
MSNTGLPKPWIVKISRSRNRPYFFNTETHESLWEPPAE

SEQ ID NO: 44 - protein, 43 aa, Saccharomyces cerevisiae
MPSDVASRTGLPTPWTVRYSKSKKREYFFNPETKHSQWEEPEG

SEQ ID NO: 45 - protein, 38 aa, Kluyveromyces lactis
MAGTGLPEPWVIKFSRSKKREYFFNPETKESVWEAPSG

SEQ ID NO: 46 - protein, 39 aa, Ashbya gossypii
MTAENGLPGPWAVKFSKSRKREYYYNPETKESQWEVPAD

SEQ ID NO: 47 - protein, 42 aa, Magnaporthe oryzae
MADQHPETGLPPNWEVRLSKSKNLPYYFNTSNTTSRWEPPPG

SEQ ID NO: 48 - protein, 39 aa, Neurospora crassa
MTIETGLPEDWEVRHSQSKNLPYYFNSATKTSRWEPPSG

SEQ ID NO: 49 - protein, 33 aa, human
AGLPPGWRKIHDAAGTYYWHVPSGSTQWQRPTW

SEQ ID NO: 50 - protein, 33 aa, mouse
TGLPPGWRKIRDAAGTYYWHVPSGSTQWQRPTW

SEQ ID NO: 51 - protein, 33 aa, rat; identical to SEQ ID NO: 50

SEQ ID NO: 52 - protein, 29 aa, human
WQEVWDENTGCYYYWNTQTNEVTWELPQY

SEQ ID NO: 53 - protein, 29 aa, mouse
WQEVWDENTGCYYYWNTQTNEVTWELPQY

SEQ ID NO: 54 - protein, 33 aa, human
SDLPAGWMRVQDTSGTYYWHIPTGTTQWEPPGR

SEQ ID NO: 55 - protein, 33 aa, mouse; identical to SEQ ID NO: 54

SEQ ID NO: 56 - protein, 33 aa, rat; identical to SEQ ID NO: 54

SEQ ID NO: 57 - protein, 33 aa, human
PDLPPGWKRVSDIAGTYYWHIPTGTTQWERPVS

SEQ ID NO: 58 - protein, 33 aa, mouse
PDLPPGWKRVNDIAGTYYWHIPTGTTQWERPVS

SEQ ID NO: 59 - protein, 35 aa, human
DPLPPGWEIKIDPQTGWPFFVDHNSRTTTWNDPRV

SEQ ID NO: 60 - protein, 35 aa, mouse; identical to SEQ ID NO: 59

SEQ ID NO: 61 - protein, 34 aa, Drosophila melanogaster
DPLPPAWNWQVTSDGDIYYYNLRERISQWEPPSP

SEQ ID NO: 62 - protein, 35 aa, Caenorhabditis elegans
QSVTLPWQRAISKSNLLPYYIEQTSEKTQWEHPVW

SEQ ID NO: 63 - protein, 35 aa, human
NATPKGWSCHWDRDHRRYFYVNEQSGESQWEFPDG

SEQ ID NO: 64 - protein, 35 aa, mouse; identical to SEQ ID NO: 63

SEQ ID NO: 65 - protein, 34 aa, human
GDNNSKWVKHWVKGGYYYYHNLETQEGGWDEPPN

SEQ ID NO: 66 - protein, 35 aa, human
ELVHAGWEKCWSRRENRPYYFNRFTNQSLWEMPVL

SEQ ID NO: 67 - protein, 35 aa, mouse
ELVHAGWEKCWSRRESRPYYFNRFTNQSLWEMPVL

SEQ ID NO: 68 - protein, 35 aa, Bos taurus
EGLPPSWYKVFDPSCGLPYYWNVDTDLVSWLSPHD

SEQ ID NO: 69 - protein, 35 aa, Gorilla gorilla
EGLPPSWYKVFDPSCGLPYYWNADTDLVSWLSPHD

SEQ ID NO: 70 - protein, 35 aa, human; identical to SEQ ID NO: 69

SEQ ID NO: 71 - protein, 35 aa, mouse
EGLPPSWYKVFDPSCGLPYYWNVETDLVSWLSPHD

SEQ ID NO: 72 - protein, 35 aa, Pongo pygmaeus; identical to SEQ ID NO: 69

SEQ ID NO: 73 - protein, 35 aa, rat; identical to SEQ ID NO: 71

SEQ ID NO: 74 - protein, 28 aa, Schizosaccharomyces pombe
WKEYATADGKKYWYNVNTRESVWDIPDE

SEQ ID NO: 75 - protein, 34 aa, human
IQINGEWETHKDSSGRCYYYNRGTQERTWKPPRW

SEQ ID NO: 76 - protein, 34 aa, Macaca fascicularis
IQINGEWETHKDSSGRCYYYDRGTQERTWKPPRW

SEQ ID NO: 77 - protein, 34 aa, mouse
IQVNGEWETHKDSSGRCYYYNRTTQERTWKPPRW

SEQ ID NO: 78 - protein, 35 aa, human
VSLETEWGQYWDEESRRVFFYNPLTGETAWEDEAE

SEQ ID NO: 79 - protein, 35 aa, mouse
ESLETEWGQYWDEESRRVFFYNPLTGETAWEDETE

SEQ ID NO: 80 - protein, 35 aa, rat
ESLETEWGQYWDEESGRVFFYNPLTGETVWEDETE

SEQ ID NO: 81 - protein, 35 aa, human
RTSENQWWELFDPNTSRFYYYNASTQRTVWHRPQG

SEQ ID NO: 82 - protein, 35 aa, mouse
RTSEDQWWELFDPNTSRFYYYSAASQRTVWHRPQN

SEQ ID NO: 83 - protein, 34 aa, human
LPLPPGWSVDWTMRGRKYYIDHNTNTTHWSHPLE

SEQ ID NO: 84 - protein, 34 aa, mouse; identical to SEQ ID NO: 83

SEQ ID NO: 85 - protein, 34 aa, rat; identical to SEQ ID NO: 83

SEQ ID NO: 86 - protein, 35 aa, Cryptococcus neoformans
IMLPQGWEARWDPQANAYIYVDQSTGRSQWEVPLN

SEQ ID NO: 87 - protein, 35 aa, Ustilago maydis
PQLPPGWVAQWDPNAARQVFVETATGRTSWQPPTA

SEQ ID NO: 88 - protein, 34 aa, human
IVLPPNWKTARDPEGKIYYYHVITRQTQWDPPTW

SEQ ID NO: 89 - protein, 30 aa, human
TPWCVVWTGDERVFFYNPTTRLSMWDRPDD

SEQ ID NO: 90 - protein, 30 aa, mouse; identical to SEQ ID NO: 89

SEQ ID NO: 91 - protein, 30 aa, human
SPWCVVWTGDDRVFFFNPTMHLSVWEKPMD

SEQ ID NO: 92 - protein, 30 aa, mouse
SPWCVVWTGDDRVFFFNPTMQLSVWEKPVD

SEQ ID NO: 93 - protein, 34 aa, chicken
EELPPGWEERTTKDGWVYYANHLEEKTQWEHPKS

SEQ ID NO: 94 - protein, 34 aa, Danio rerio
DELPPGWEERSTKDGWVYYANHEEMKTQWEHPKT

SEQ ID NO: 95 - protein, 34 aa, human
DELPPGWEERTTKDGWVYYANHTEEKTQWEHPKT

SEQ ID NO: 96 - protein, 34 aa, mouse; identical to SEQ ID NO: 95

SEQ ID NO: 97 - protein, 34 aa, Pongo abelii; identical to SEQ ID NO: 95

SEQ ID NO: 98 - protein, 35 aa, yeast
PQVPSGWKAVFDDEYQTWYYVDLSTNSSQWEPPRG

SEQ ID NO: 99 - protein, 35 aa, Arabidopsis thaliana
HTLPKPWKGLIDDRTGYLYFWNPETNVTQYEKPTP

SEQ ID NO: 100 - protein, 35 aa, Oryza sativa
PTLPKPWRGLIDGNTGYLYFWNPETKAVQYDRPTA

SEQ ID NO: 101 - protein, 35 aa, Arabidopsis thaliana
PTLPQPWKGLIDGSTGILYYWNPETNVTQYERPSA

SEQ ID NO: 102 - protein, 35 aa, Oryza sativa
PSLPKPWRGLVDGTTGYLYYWNPETNITQYEKPLP

SEQ ID NO: 103 - protein, 35 aa, Arabidopsis thaliana
PNLPKPWKGLVDSRTGYLYFWNPETNVTQYERPAS

(19) United States
(12) Patent Application Publication
(10) Pub. No.: US 2014/0005126 A1
(43) Pub. Date: Jan. 2, 2014

(54) SCAFFOLD PEPTIDES

(71) Applicants: Chris Ullman, Cambridge (GB); Agnes Jaulent, Cambridge (GB); Seema Patel, Cambridge (GB); Pascale Mathonet, Cambridge (GB)

(72) Inventors: Chris Ullman, Cambridge (GB); Agnes Jaulent, Cambridge (GB); Seema Patel, Cambridge (GB); Pascale Mathonet, Cambridge (GB)

(73) Assignee: ISOGENICA LTD, Cambridge (GB)

(21) Appl. No.: 13/814,536

(22) PCT Filed: Aug. 8, 2011
(86) PCT No.: PCT/GB11/51500

§ 371 Date: Sep. 9, 2013

(30) Foreign Application Priority Data
Aug. 6, 2010 (GB) 1013284.3

(51) Int. Cl. C07K 14/47

(52) U.S. Cl. CPC C07K 14/47
USPC 514/21.3; 506/18; 530/300; 530/324; 530/391.7; 530/317; 435/375

(57) ABSTRACT

A naive WW domain peptide library derived from a WW domain peptide sequence which has been diversified by changing the amino acid sequence at one or more positions is provided. The naive WW domain peptide library may be derived from a GroupIV WW domain peptide. Methods for making the naive WW domain peptide library and methods for selected a modified WW domain peptide that binds a target ligand using the naive WW domain peptide library are also provided. Also disclosed are modified WW domain peptides that bind desired target ligands, pharmaceutical compositions comprising such peptides, and uses for such peptides. The modified WW domain peptides have altered, improved or different, target ligand binding characteristics to those of the unmodified WW domain peptides from which they are derived.

### FIELD OF THE INVENTION

This invention relates to novel polypeptide scaffolds for the selection of polypeptides having desirable functions, such as binding affinity to target ligands, and also to methods of using the scaffolds, and resulting polypeptides. In particular, the invention relates to naïve libraries based on the WW domain, to methods of selecting novel binding moieties from the libraries, and to functional modified WW domain peptides.

### BACKGROUND OF THE INVENTION

Proteins exist in a variety of different shapes that interact with each other and other molecules to direct natural signalling events and enzymatic reactions. Many proteins are folded into defined three-dimensional structures which have been classified in a database (Murzin A. G. et al., (1995) SCOP: a structural classification of proteins database for the investigation of sequences and structures, J. Mol. Biol. 247, 536-540 [http://scop.mrc-lmb.cam.ac.uk/scop/]). Some proteins (and families of proteins) have been subject to extensive engineering in order to produce altered activities and, hence, new uses. Typically, only select regions of a protein are modified, so that the protein engineer can retain desirable properties of the natural protein, such as folding, and certain functionality. The regions that are not modified (i.e. the constant regions) may, therefore, be considered to represent the natural protein's backbone, scaffold or framework. It is often advantageous if these unmodified wild-type regions are those that determine the three-dimensional folding of the protein, so as to maintain the shape/configuration of the protein (or peptide) of interest and provide the best opportunity for creating a functional mutant protein. The regions, domains or loops in the protein that are less critical for the protein's structure can then be modified or even randomised to change the functionality of the wild-type protein and, hopefully, to obtain new and useful properties.

The technique of using a protein “scaffold” and engineering of loops or regions within the scaffold to alter activity is most notable with regard to the field of antibodies and antibody fragments, which have a natural repertoire of variable regions or loops. The variable loops of antibodies have been extensively engineered to produce peptides having improved binding (e.g. affinity and/or specificity) to known ligands, and also to expand the binding substrates for particular antibody frameworks (see for example, Knappik et al., (2000) J. Mol. Biol., 296, 57-86; and EP 1025218). The engineering of non-antibody frameworks has been reviewed, for example, by Hosse et al., (2006), Protein Sci., 15, 14-27.

WW domains are small peptide domains that are found naturally as part of much larger proteins. They have been shown to bind linear peptide sequences involved in protein-protein interactions (see Staub & Rotin, (1996) Structure, 4, 495-499 for a review). Since WW domains are relatively small structures, they have served as good subjects for understanding the folding energetics of small beta-sheet proteins. Several structures of single or paired WW domains have been determined, both with and without a bound ligand (Wintjens et al., (2001) J. Biol. Chem., 276, 25150-25156; Kanelis et al., (2006) Structure, 14, 543-553; Weisner et al., (2002) J. Mol. Biol., 324, 807-822; Macias et al., (2002) FEBS Lett., 513, 30-37), and these have shown that the domain adopts an anti-parallel three-stranded beta-sheet (or ‘beta meander’) with a cup-shaped binding surface. The name ‘WW domain’ derives from the fact that a pair of conserved tryptophan (W) residues is known to be essential for the formation of the structure. However, as ever, there are rare exceptions to this rule, where phenylalanine or tyrosine are found to replace one of the tryptophan residues.

The binding specificity of the WW domain has led to the classification of these domains into five groups dependent upon the ligand sequence. Group I domains recognise PPXY motifs in the ligand; Group II domains recognise PPLP motifs; Group III domains bind proline-rich stretches in methionine and glycine (PGM motif); Group IV domains bind phosphoserine and phosphothreonine residues; and Group V domains bind stretches of proline that are rich in arginine (Macias et al., (2002) FEBS Lett., 513, 30-37). More recently, 42 WW domains have been studied using NMR and with peptide library screens, which has led to the classification of 32 WW peptides into six groups based upon their recognition of proline and phosphoserine and threonine peptide motifs. Further analysis of the human Pin1 WW domain, a Group IV domain, with 600 potential tyrosine, serine and threonine kinase target sequences, demonstrated that this domain binds exclusively to phosphorylated serine/threonine targets (poS/poT), and not to phosphotyrosine peptides or non-phosphorylated target sequences (Otte et al., (2003) Protein Science, 12, 491-500). In fact, binding was found to be strongest with poT peptides of the sequence φφppoTPP (where φ is a hydrophobic residue and P is proline).

Various mutagenesis studies have been carried out to investigate the structure and function of WW domains. In studies of the Smurf2 WW domain a non-canonical phenylalanine residue was found to reduce its affinity for PY peptide, although interactions between the PY tail and the β1 and β1-2 loops of Smurf2 were shown to contribute to binding (Chong et al., (2006) J. Biol. Chem., 281, 17069-17075). In other studies, mutations to various amino acids within WW domains have been shown to provide modified properties, such as: (1) improved stability—Jiang et al., (2001) Protein Science, 10, 1454-1465)—in this study of the human Yes-associated domain (hYAP), three positions within the domain were mutated (A20R/L30Y/D34T) to reflect the sequences of the WW domains from Pin1 and FBP28; (2) improved stability—Jäger et al., (2007) Protein Science, 16, 2306-2313)—in this study the wild-type Pin1 sequence was modified by introduction of a single or a pair of tryptophan residues, which increased the thermostability of the domain, but reduced the binding affinity for its natural peptide ligand to below detectable limits; and (3) improved stability—Jäger et al., (2006) PNAS, 103, 10648-10653)—the length of loop1 in Pin1 was reduced by one amino acid, by substituting its native loop with the sequence of loop1 from FBP WW domain. The stability of the domain was improved but, again, the natural peptide ligand was not bound; (4) altered binding specificity—Kasanov et al., (2001) Chemistry & Biology, 8, 231-241)—in this study the amino acid sequence of the Nedd4.3 domain (a Group I WW domain) was changed at positions D24, H32, T37 so that the domain bound different peptide motifs.

In another study the Pin1 domain was mutated in order to modulate its binding to phosphorylated ligands (WO/2000/048621), and it was concluded that the Pin1 domain binds serine or threonine phosphoproteins or polypeptides with high affinity in a phosphate dependent manner. It is thought that Pin1 modulates its ligand by interacting with the regulatory domain of the ligand, where the regulatory domain undergoes alterations (e.g. in phosphorylation status) during cellular/biological processes.

Other engineering projects include the hYAP WW domain (a Group I domain) which has been mutated and displayed on the surface of phage particles in order to select for improved binding to a known peptide ligand containing the consensus motif PPXY (Dalby et al., (2000) Protein Science, 9, 2366-2376). In addition, studies of the amino acid sequences of 292 WW domains has also led to the identification of coevolving contact residues L₄P₅G₆E₈F₂₁N₂₂H₂₃S₂₈ (Russ et al., (2005) Nature, 437, 579-583; and Socolich et al., (2005) Nature, 437, 512-518) and in silico design of new domains for binding to peptide libraries containing the consensus peptide binding motifs. No Group II or IV domains (Pin1) were identified.

Despite the number of earlier mutagenesis experiments that have been performed on WW domains, none of these studies has shown that the WW domain can be engineered to bind natural extracellular ligands that were previously not recognised by the wild-type WW domain sequence. Many applications can be envisaged, however, for mutated WW domains having altered binding specificities. Accordingly, it would be desirable to be able to engineer WW domains to have de novo binding specificities for previously unrecognised ligands, for example, for use in therapy or as diagnostic tools. It would be particularly advantageous to provide a modified WW domain framework or scaffold for use in the design of new ligand binding domains having desired binding specificities and/or affinities, for example, to act as a research tool for the design, selection and screening of new and useful binding domains.

Furthermore, in all of the above studies, where binding affinities have been measured (e.g. by tryptophan fluorescence emission spectroscopy or isothermal calorimetry) and reported, for either natural or mutant WW domains and their target ligand, the affinities have invariably been found to be greater than 1 μM. For example, the affinities of wild-type Pin1 WW domain against its natural target ligands were measured at between 7.7 and 126 μM (Verdecia et al., (2000) Nature Structural Biol., 7, 639-643). Such binding affinities are lower (weaker) than is typically desired for use in diagnostic tools or as therapeutics. Hence, it would also be advantageous to have a WW domain peptide scaffold and screening system for designing and selecting modified WW domain (peptide)-binding domains having high affinity for both new and wild-type ligands. It would also be desirable to have modified WW domain peptides having such high binding affinities against desired target ligands.

For many applications that can be envisaged for designer WW domains for use as therapeutics, diagnostic tools and so on, it would be beneficial to have greater freedom to select a desired target ligand, for example, without the limitation/requirement of having a phosphorylated serine or threonine residue in the recognition sequence. Moreover, for many applications it would be an advantage to have a human derived binding domain scaffold and associated binding domains. Hence, it would be desirable to have a Pin1 WW domain framework for the design and selection of new binding domains, particularly engineered high-affinity Pin1 WW domains (e.g. with binding affinities in the order of nMs), and more particularly, engineered Pin1 WW domains that recognise non-phosphorylated ligands.

Accordingly, the present invention seeks to overcome or at least alleviate one or more of the problems in the prior art.

### SUMMARY OF THE INVENTION

In general terms, the present invention provides a modified, stabilised WW domain framework or scaffold, which can be used for the selection of de novo binding domains having desired binding characteristics, such as affinity for new target molecules and/or high affinity for known or new ligands. The modified WW domain framework is a relatively compact and self-contained unit which may provide a universal template for the design and selection of new binding molecules that are stable, have suitably high affinity for their target ligands, can be readily synthesised, and/or may be useful in therapeutic and non-therapeutic applications. For example, the modified WW domain framework may have the same or similar applications to those of engineered antibodies, antibody peptides and antibody fragments. Furthermore, the invention relates to methods for the selection of modified WW domains that have one or more desirable activity, such as binding affinity for new target molecules/ligands, e.g. peptide sequences. In addition, the invention relates to modified WW domains having the above-mentioned desirable activities, to compositions comprising such modified WW domains and to therapeutic and diagnostic molecules and compositions comprising modified WW domains identified and/or derivatised in accordance with the invention. In some aspects and embodiments, the invention is directed to modified Pin1 WW domain frameworks and to modified Pin1 WW domain peptides produced using the methods of the invention.

Hence, in one aspect of this invention, the Applicant has created libraries of the Pin1 WW domain, which comprise a framework or backbone structure of predominantly wild-type amino acid residues, which acts as a structural and functional template to support regions and/or positions of variable amino acids, which provide for the selection of WW domains having new properties/activities, such as new ligand recognition motifs. These modified frameworks or libraries may be subjected to a selection process to isolate individual modified WW peptide domains having a particular desired activity. In this way, novel WW domains based on Pin1 have been identified that bind non-phosphorylated target peptide sequences, and which bind entirely new peptide sequences compared to the natural ligand for Pin1. These modified WW domain peptides are particularly interesting for their potential utility in therapy and also in non-therapeutic applications. Compared with many known peptide therapeutic molecules the modified WW domains of the invention are advantageous because they are relatively short and stable peptide domains, and have a relatively simple fold. Modified WW domain peptides of the invention may be cyclised, for example, to provide increased stability for therapeutic and other in vivo applications, and such cyclised peptides have been demonstrated to maintain the desired target binding activity.

Accordingly, in a first aspect of the invention there is provided a naïve WW domain peptide library which has a consensus sequence derived from a WW domain peptide sequence which has been diversified by changing the amino acid sequence at one or more positions. The framework of the naïve WW domain peptide library may be derived from a wild-type WW domain. In one embodiment the consensus sequence has at least three invariant tryptophan residues. By “invariant” it is meant that the tryptophan residues are within the framework (i.e. non-variable) part of the WW domain library. The invariant tryptophan residues may be derived from the parent/wild-type WW domain peptide sequence or may be artificially introduced. Additional tryptophan residues may be included within the naïve/variable sequence of the library. Suitably, substantially all functional members of the library have a three-stranded beta-sheet fold, which may be an anti-parallel beta-sheet. In some embodiments, the naïve WW domain peptide library is derived from a Group IV WW domain sequence, suitably from a human WW domain sequence, and more suitably from a Pin1 WW domain peptide sequence. In a particularly suitable embodiment the naïve WW domain peptide library is derived from the amino acids at positions 6 to 38 of SEQ ID NO: 1. The naïve WW domain peptide library of the invention may include at least one amino acid deletion relative to the sequence from which it is derived. In another embodiment the naïve WW domain peptide library of the invention may include at least one amino acid insertion relative to the sequence from which it is derived. Preferably the deletion and/or insertion is in a loop region of the WW domain. A most suitable naïve WW domain peptide library is derived from the amino acid sequence at positions 6 to 37 of SEQ ID NO: 2; wherein at least the amino acids at positions X₁ to X₉ are variable. Preferably, the naïve WW domain peptide library comprises the amino acid sequence at positions 6 to 37 of SEQ ID NO: 2, wherein positions X₁ to X₉ are variable. Preferred sub-groups of amino acids for incorporation at positions X₁ to X₉, depending on the target ligand, are also given in the Examples.

In another aspect the invention relates to the use of a naïve WW domain peptide library of the invention in the selection of a modified WW domain peptide against a target ligand. Suitably, the naïve WW domain peptide library has no pre-determined target ligand specificity. In this any other aspects of the invention, the naïve WW domain peptide library may be derived by mutating a wild-type WW domain sequence at one or more amino acid positions. Advantageously, in this any other aspect, the target ligand may be a ligand that is not bound by the wild-type WW domain peptide

Thus, another aspect of the invention relates to a modified WW domain peptide identified from the libraries and methods of the invention. The modified WW domain peptide of the invention is suitably derived from a wild-type WW domain peptide sequence which has been diversified by changing the amino acid sequence at one or more positions. Optionally, the modified WW domain peptide binds a target ligand not bound by the wild-type WW domain peptide from which it is derived. Suitably, no more than 70%, no more than 60%, no more than 50%, no more than 40%, or no more than 30% of the amino acids of the wild-type sequence are changed. In this way, the wild-type WW domain framework can be readily identified. In one embodiment, the modified WW domain peptide of the invention has a consensus sequence comprising the amino acid sequence WX₃WX₁₆₋₁₈W where X is any amino acid. In another embodiment, the modified WW domain peptide of the invention has a consensus sequence comprising the amino acid sequence WX₃WX₁₈₋₃₂W wherein X is any amino acid. Thus, the consensus sequence may comprise the amino acid sequence WX₃WX₁₆₋₃₂W. It will be appreciated that since the modified WW domain of the invention may be derived from the naïve WW domain libraries of the invention, they may have any one or more of the features of the naïve WW domain library peptides. Beneficially, the modified WW domain peptide of the invention binds to a target ligand that is not bound by the WW domain peptide from which it is derived. Alternatively, the modified WW domain peptide of the invention binds the natural ligand of the non-modified WW domain with greater binding affinity than the wild-type peptide. In a particularly suitable embodiment, the modified WW domain peptide binds a non-phosphorylated ligand and the WW domain peptide from which it is derived does not bind to non-phosphorylated ligands. Suitable, the non-phosphorylated ligand is an extracellular target sequence in vivo. Advantageously, the modified WW domain peptide of the invention binds its target ligand with a dissociation constant (Kd) of less than 1 μM, less than 200 nM, or less than 100 nM. Suitably target ligands for the modified WW domain peptides of the invention include VEGFR2 and NGF. Preferred modified WW domain peptides of the invention comprise the amino acid sequence at positions 6 to 38 of SEQ ID NOs: 15 to 20 and 26 to 29 (numbering in accordance with the sequence of SEQ ID NO: 1). A most suitable modified WW domain peptide is cyclised to create a cyclic peptide, which may be preferred for therapeutic and/or in vivo applications.

Accordingly, the invention encompasses therapeutic and diagnostic uses for the modified WW domain peptides of the invention. Aspects and embodiments of the invention therefore include formulations, medicaments and pharmaceutical compositions comprising the modified WW domain peptides. In one embodiment the invention relates to a modified WW domain peptide for use in medicine. More specifically, for use in antagonising or agonising the function of a target ligand, such as an extracellular target ligand. The modified WW domain peptides of the invention may be used in the treatment of various diseases and conditions of the human or animal body, such as cancer, degenerative disease of the retina, or pain. Treatment may also include preventative as well as therapeutic treatments and alleviation of a disease or condition.

The invention further encompasses nucleic acids, such as expression vectors, that encode the naïve WW domain peptide libraries of the invention, or the modified WW domain peptides of the invention, or WW domain peptides obtainable by the methods of the invention.

In yet another aspect of the invention, there is provided a method of making a naïve WW domain peptide library, the method comprising: (a) providing a plurality of nucleic acids each encoding a WW domain peptide; (b) introducing diversity into the plurality of nucleic acids, thereby to create a plurality of modified nucleic acids, to provide diversity at one or more amino acid residues in the WW domain peptide of a plurality of said WW domain peptides encoded by the modified nucleic acids; and (c) expressing the WW domain peptides encoded by said plurality of modified nucleic acids, whereby a library of modified WW domain peptides comprising sequence diversifications is produced. In some embodiments, step (b) of the method may further comprise amplifying the nucleic acid sequence, e.g. using PCR. In one embodiment the method may further comprise: (d) selecting peptides of the library against a target ligand against which the WW domain peptides in step (a) have not been selected. The target ligand may not be bound by the (non-modified) WW domain peptides in step (a). The method of this aspect of the invention may further comprise isolating desirable modified WW domains. In one embodiment the method comprises: (e) isolating peptides that bind to the target ligand with a dissociation constant (Kd) of less than 1 μM, less than 200 nM, or less than 100 nM.

In yet another aspect the invention provides a method for isolating a modified WW domain peptide from a naïve WW domain peptide display library, the library comprising a plurality of nucleic acid sequences that encode displayed modified WW domain peptides, comprising the steps of: (a) expressing a plurality of nucleic acid constructs, wherein each nucleic acid construct comprises a promoter sequence operably linked to the nucleic acid sequence, such that expression of the plurality of nucleic acid constructs results in formation of a plurality of peptide-nucleic acid complexes, each complex comprising at least one displayed modified WW domain peptide associated with the corresponding nucleic acid construct encoding the displayed peptide; (b) exposing the plurality of peptide-nucleic acid complexes to at least one target ligand, and allowing the peptide-nucleic acid complexes to associate with the ligand, suitably by binding of a displayed modified WW domain peptide to the target ligand; (c) removing any peptide-nucleic acid complexes that remain unassociated with the target ligand; and (d) recovering any target ligand-associated peptide-nucleic acid complexes. In one embodiment the method further comprises: (e) characterising the peptide encoded by the nucleic acid sequence of any recovered ligand-associated peptide-nucleic acid complexes as comprising a target ligand-binding modified WW domain peptide. Suitably, the peptide display library is an in vitro peptide display library, and most suitably a CIS in vitro peptide display library. The target ligand may be defined as disclosed elsewhere herein, such as in relation to any of the above aspects of the invention. The method may further comprise one or more of the following: (A) correlating one or more target ligand-binding modified WW domain peptide of step (e) with the corresponding nucleic acid construct, thereby identifying nucleic acid sequences for the one or more target ligand-binding modified WW domain peptide; (B) isolating a modified WW domain peptide that associates with the target ligand; (C) forming a derivative of the modified WW domain peptide, optionally wherein the derivative is formed by performing a maturation experiment to improve one or more characteristics of the modified WW domain peptide; (D) conjugating the modified WW domain peptide to another moiety, such as a non-WW domain moiety; (E) isolating the nucleic acid construct that encodes the modified WW domain peptide of any recovered target ligand-associated peptide-nucleic acid complexes, and optionally inserting it into an expression vector or construct; and/or (F) formulating a pharmaceutical composition comprising the modified WW domain peptide of the invention, and/or a nucleic acid construct encoding the modified WW domain peptide of the invention.

It will be appreciated that modified WW domain peptides of the invention may be further derivatised or conjugated to additional molecules and that such derivatives and conjugates fall within the scope of the invention.

It should also be appreciated that, unless otherwise stated, optional features of one or more aspects of the invention may be incorporated into any other aspect of the invention and that all such variations are encompassed within the scope of the invention.

All references cited herein are incorporated by reference in their entirety. Unless otherwise defined, all technical and scientific terms used herein have the same meaning as commonly understood by one of ordinary skill in the art to which this invention belongs.

### BRIEF DESCRIPTION OF THE DRAWINGS

The invention is further illustrated by the accompanying drawings in which:

FIG. 1 illustrates the high-resolution surface structure of the WW domain of Pin1 indicating the putative ligand-binding surface;

FIG. 2 shows the results of an ELISA screen of modified WW domain peptides selected to bind to VEGFR2. The dark grey bars show the signal obtained for selected peptides binding to VEGFR2 coated wells, whereas the light grey bars represent the signal obtained in a control using uncoated wells. The ELISA signal was read at 450 nm.

FIG. 3 shows a sequence alignment of the WW domain peptides selected to bind to VEGFR2, which were analysed by ELISA (as illustrated in FIG. 2). An X in the Pin1 peptide domain sequence illustrates the residues that were diversified by mutation in the naïve WW domain library.

FIG. 4 demonstrates the specificity of the modified WW domain peptides selected to bind to VEGFR2 as assessed by ELISA assay. The modified WW domain peptides were tested against a plurality of target and non-target ligands: VEGFR2 (dark grey column); human IgG (narrow cross hatch column); a monoclonal antibody (speckled column); an enzyme (light grey column); and human serum albumin (wide crosshatch column). The white column is a blocked well control.

FIG. 5 shows the steady state affinity measurements for the binding of linear WW-B1 to VEGFR2 as measured by biolayer interferometry.

FIG. 6 shows a competition assay for the binding of VEGF to VEGFR2 in the presence of a selected modified WW domain peptide, clone WW-B1, chemically synthesised.

FIG. 7 is an ELISA screen of modified WW domain peptides selected to bind to NGF. The black bars show the signal obtained for selected peptides binding to NGF coated wells, whereas the light grey bars represent the signal obtained in a control using HSA coated wells. The ELISA signal was read at 450 nm.

FIG. 8 shows a sequence alignment of the WW domain peptides selected to bind to NGF, which were analysed by ELISA (as illustrated in FIG. 7). An X in the Pin1 peptide domain sequence illustrates the residues that were diversified by mutation in the naïve WW domain library.

FIG. 9 shows the MALDI-TOF mass spectrometry results for a cyclic WW-B1 peptide (Glu38 variant), cyclised through amino acids at positions 6 and 38 (numbering according to SEQ ID NO: 1), showing the correct expected mass.

FIG. 10 shows the results of an analytical reverse phase HPLC trace of the cyclic WW-B1 peptide (Glu38 variant), cyclised through amino acids at positions 6 and 38 (numbering according to SEQ ID NO: 1), using a RP C18 column.

FIG. 11 shows the steady state affinity measurements for the binding of cyclic WW-B1 (Glu38 variant), cyclised through amino acids at positions 6 and 38 (numbering according to SEQ ID NO: 1), to VEGFR2 as measured by biolayer interferometry.

FIG. 12 shows the steady state affinity measurements for the binding of cyclic WW-B1 (Glu38 variant), cyclised through amino acids at positions 6 and 38 (numbering according to SEQ ID NO: 1) to VEGFR2 as measured by biolayer interferometry.

FIG. 13 shows the MALDI-TOF mass spectrometry results for a cyclic B1 peptide (Glu38 variant), cyclised through amino acids at positions 4 and 29 (numbering according to SEQ ID NO: 1), showing the correct expected mass.

FIG. 14 shows the plot of the area under the curve for peptide WW-B1, as analysed by HPLC, following incubation in mouse plasma for up to 20 hours.

FIG. 15 shows the far-UV CD spectra for A: WW-B1; and B: cyclic WW-B1 (Glu38 variant) cyclised through amino acids at positions 6 and 38. The dotted line represents the CD spectra measured at 25° C.; the solid line represents the CD spectra measured at 90° C. and the open circles represents the CD spectra of the peptide heated to 95° C. and then cooled to 25° C.

### DETAILED DESCRIPTION OF THE INVENTION

In order to assist with the understanding of the invention several terms are defined herein.

The term “peptide” as used herein (e.g. in the context of a WW domain peptide framework/template/scaffold, or a modified WW domain peptide) refers to a plurality of amino acids joined together in a linear or circular chain. The term oligopeptide is typically used to describe peptides having between 2 and about 50 or more amino acids. Peptides larger than about 50 are often referred to as polypeptides or proteins. For purposes of the present invention, the term “peptide” is not limited to any particular number of amino acids. Preferably, however, they contain up to about 100 amino acids, up to about 70 residues, or up to about 50 residues. Suitably, a modified WW domain peptide of the invention contains between about 20 and about 50 amino acid residues and more suitably between about 22 and about 45 residues. In some embodiments a modified WW domain framework or peptide may contain about 23 to about 40 amino acid residues, or between about 26 and about 39 residues: for example, 30, 31, 32, 33, 34, 35 or 36 amino acids. It should be understood that an isolated WW domain framework or modified peptide of the invention may comprise or consist of the above number of amino acids.

The term “amino acid” in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

A “modified WW domain” (peptide) of the invention is a based on a wild-type WW domain that has been mutated (e.g. amino acid substitution, deletion, addition) in at least one position. Thus, the modified WW domain is conveniently derived from a wild-type protein or peptide sequence. Typically, it is derived from a fragment of a wild-type protein, e.g. Pin1. It will be appreciated that, depending on the application, the modified WW domain peptide of the invention may comprise an additional peptide sequence or sequences at the N- and/or C-terminus in comparison to the corresponding wild-type peptide sequence from which it is derived: for example, the dipeptide sequence met-ala may be included at the N-terminus for ease of protein expression and/or nucleic acid cloning. Where the Met-Ala dipeptide is indicated at the N-terminus of the wild-type Pin1 WW domain sequence, a modified Pin1 WW domain peptide sequence, and in the sequences of WW domain frameworks, it should be appreciated that this dipeptide part of the wild-type sequence, but corresponding sequences lacking the Met-Ala N-terminal dipeptide are considered to be encompassed within the scope of the invention. An alignment of Pin domains from different organisms is shown in Table 1, which demonstrates the diversity of sequence in this domain especially at the N-terminus of the domain. The alignment of Table 1 also demonstrates how the numbering of the amino acids in Pin1 can be used as a reference to other WW domain peptides that may be used in accordance with the invention.

TABLE 1
Alignment Of WW Pin Domains
(from top to bottom: SEQ ID NOs: 1 and 35 to 48, respectively).

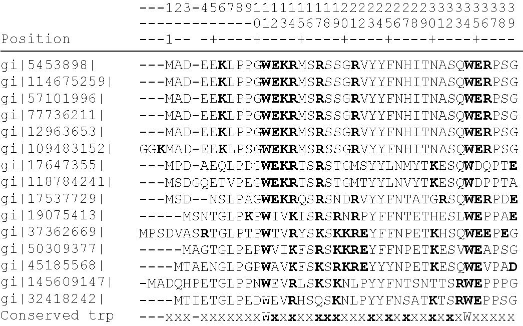

Key
gi|5453898|ref|NP_006212.1| peptidyl-prolyl cis-trans isomerase
NIMA-interacting 1 [Homo sapiens] SEQ ID NO: 1
gi|114675259|ref|XP_001161914.1| PREDICTED: similar to Chain B, Structural
Basis For The Phosphoserine-Proline Recognition By Group IV WW Domains
isoform 1 [Pan troglodytes] SEQ ID NO: 35
gi|57101996|ref|XP_542080.1| PREDICTED: similar to Peptidyl-prolyl cis-trans
isomerase NIMA-interacting 1 [Canis familiaris] SEQ ID NO: 36
gi|77736211|ref|NP_001029804.1| peptidyl-prolyl cis-trans isomerase NIMA-
interacting 1 [Bos taurus] SEQ ID NO: 37
gi|12963653|ref|NP_075860.1| peptidyl-prolyl cis-trans isomerase NIMA-
interacting 1 [Mus musculus] SEQ ID NO: 38
gi|109483152|ref|XP_216609.3| PREDICTED: similar to protein [Rattus
norvegicus] SEQ ID NO: 39
gi|17647355|ref|NP_523428.1| dodo [Drosophila melanogaster] SEQ ID NO: 40
gi|118784241|ref|XP_313593.3| AGAP004321-PA [Anopheles gambiae str. PEST]
SEQ ID NO: 41
gi|17537729|ref|NP_494393.1| hypothetical protein Y110A2AL.13
[Caenorhabditis elegans] SEQ ID NO: 42
gi|19075413|ref|NP_587913.1| peptidyl-prolyl cis-trans isomerase Pin1
[Schizosaccharomyces pombe] SEQ ID NO: 43
gi|37362669|ref|NP_012551.2| Ess1p [Saccharomyces cerevisiae S288c]
SEQ ID NO: 44
gi|50309377|ref|XP_454696.1| unnamed protein product [Kluyveromyces lactis]
SEQ ID NO: 45
gi|45185568|ref|NP_983284.1| ACL120Wp [Ashbya gossypii ATCC 10895]
SEQ ID NO: 46
gi|145609147|ref|XP_367478.2| hypothetical protein MGG_07389 [Magnaporthe
oryzae 70-15] SEQ ID NO: 47
gi|32418242|ref|XP_329599.1| hypothetical protein [Neurospora crassa]
SEQ ID NO: 48

Modified WW domain peptides of the invention typically contain naturally occurring amino acid residues, but in some cases non-naturally occurring amino acid residues may also be present. Therefore, so-called “peptide mimetics” and “peptide analogues”, which may include non-amino acid chemical structures that mimic the structure of a particular amino acid or peptide, may also be used within the context of the invention. Such mimetics or analogues are characterised generally as exhibiting similar physical characteristics such as size, charge or hydrophobicity, and the appropriate spatial orientation that is found in their natural peptide counterparts. A specific example of a peptide mimetic compound is a compound in which the amide bond between one or more of the amino acids is replaced by, for example, a carbon-carbon bond or other non-amide bond, as is well known in the art (see, for example Sawyer, in Peptide Based Drug Design, pp. 378-422, ACS, Washington D.C. 1995). Such modifications may be particularly advantageous for increasing the stability of modified WW domains and/or for improving or modifying solubility, bioavailability and delivery characteristics (e.g. for in vivo applications).

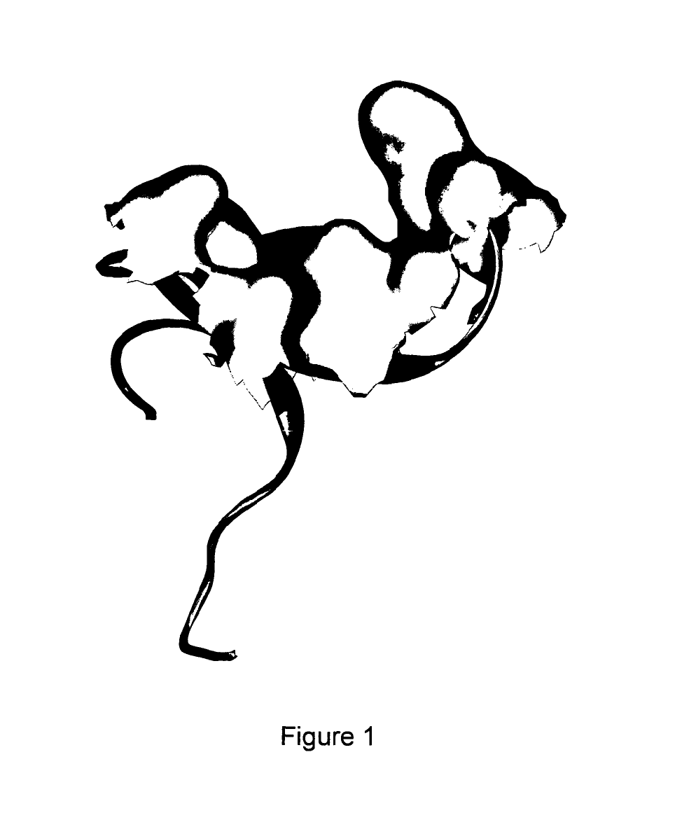

One aspect of the present invention is directed towards a WW domain framework, scaffold or template (which terms are used interchangeably herein), which can be used to create libraries of modified WW domain peptides for screening to identify modified WW domains having desirable physical properties and characteristics. It will be understood that the WW domain framework may be a nucleic acid sequence or a peptide sequence. The WW domain framework of the invention is derived from a suitable wild-type WW domain peptide, such as the Pin1 WW domain. Thus, it comprises a core or backbone of amino acid residues of the wild-type peptide from which it is derived, with a plurality of amino acid mutations (e.g. substitutions) at various positions in comparison to the corresponding wild-type sequence. Conveniently, therefore, a “WW domain framework” as used herein encompasses a library (or population) of different but related WW domain peptides based around a common core sequence with specific or random mutations at one or more positions within the domain; and as such may also be termed a WW domain framework (nucleic acid or peptide) library. Such a library having a mixture of peptides or nucleic acids that has not been optimised or selected to have a particular functionality is termed herein a “naïve” library. An individual peptide expressed from a WW domain framework library of the invention is therefore considered to be a “modified WW domain peptide” as discussed above, and its encoding nucleic acid can be considered a “modified WW domain nucleic acid”. Beneficially, although not exclusively, a modified WW domain peptide adopts the characterised WW domain protein fold comprising a three-stranded β-sheet or β-meander. Likewise, the WW domain framework peptide may (although not necessarily exclusively), form a cup-shaped binding surface for receiving and binding target ligands. FIG. 1 shows the cup-shaped binding surface of a WW domain peptide (figure adapted from PDB:1l6C structure determined by Wintjens et al., (2001), J. Biol. Chem. 276, 25150-25156). A potential advantage of the WW domain framework of the invention is that target ligands that may be bound by individual members of the particular framework library may not be restricted to a particular type or conformation of molecule (e.g. linear peptide). Thus, any desirable ligand may be recognised (i.e. bound) by WW domains from the WW domain framework library, such as nucleic acids (e.g. DNA or RNA), small organic or inorganic molecules, proteins or peptides. A suitable ligand is a protein, and a particularly suitable ligand is a peptide sequence or “epitope” of a protein. A preferred target ligand is a linear peptide, which may be isolated or part of a larger peptide or protein molecule.

Another aspect of the present invention is directed towards the identification and characterisation of modified WW domains having a desired property, from amongst a population (or library) of mutant WW domains based on a WW domain framework. The library comprises a plurality of nucleic acid sequences (e.g. at least 10⁶, 10⁸, 10⁹, 10¹² or more different coding sequences) that can be expressed and screened to identify modified WW domains having the desired property.

Typically, the modified WW domain is derived from the Pin1 WW domain peptide sequence, i.e. N′-MADEEKLPPGWEKRMSRSSGRVYYFNHITNASQWERPSG-C′ (SEQ ID NO: 1), which is a peptide fragment of the human Pin1 protein. The modified WW domain may thus be selected from a library of mutant Pin1 WW domain peptides (or fragments of a mutated Pin1 protein). A selected modified Pin1 WW domain of the invention contains 1 or more, suitably 3 or more, 5 or more, or 7 or more mutations relative to the wild-type Pin1 WW domain sequence from which it is derived. However, it is beneficial that the modified Pin1 WW domain peptide is at least 30%, at least 50%, or at least 70% identical to the corresponding wild-type sequence. The modified WW domain of the invention is most preferably a three-stranded beta sheet. Advantageously, the modified WW domain comprises at least 3 tryptophan residues, two of which are located at the corresponding position to those of the wild-type sequence or library from which the modified domain is derived (where the natural sequence includes 2 tryptophan residues). Still more preferably, the modified WW domain further comprises Asn26 and contains a minimum of 3 tryptophan residues. Thus, in preferred embodiments, the wild-type Pin1 WW domain peptide sequence is diversified by changing up to 15 amino acid positions, up to 12 amino acid positions, or up to 10 amino acid positions but includes at least 3 tryptophan residues. A preferred form of mutation is an amino acid substitution. Alternatively, the modified WW domain may be derived from other WW domain-containing proteins derived from humans or other animals, such as mammals and other eukaryotes such as birds, amphibians, insects, worms, fungi, yeast, and plant species (see Table 2 for alternative WW domain peptide sequences that may be modified and used to create scaffolds of the invention). In particular, WW domain peptide fragments containing at least three invariant tryptophan residues may also be used as a starting point for selection of modified WW domains of the invention.

TABLE 2
Representative alignment of WW domains with 3 or more Trp residues or that
have a conserved Trp residue in an unusual position and a conservative Trp/Tyr
substitution (from top to bottom: SEQ ID NOs: 49 to 103 respectively).

| | | Number | |
|---|---|---|---|
| Protein accession | Amino acid sequence alignment | of Trp | SeqID |
| APBB3_HUMAN/29-61 | AGLPPGWRKIHDAAG.-TYYWHVPSGSTQWQRPTW | 4 | 49 |
| APBB3_MOUSE/29-61 | TGLPPGWRKIRDAAG.-TYYWHVPSGSTQWQRPTW | 4 | 50 |
| APBB3_RAT/29-61 | TGLPPGWRKIRDAAG.-TYYWHVPSGSTQWQRPTW | 4 | 51 |
| FNBP4_HUMAN/220-248 | WQEVWDENTgCYYYWNTQTNEVTWELPQY | 4 | 52 |
| FNBP4_MOUSE/224-252 | WQEVWDENTgCYYYWNTQTNEVTWELPQY | 4 | 53 |
| APBB1_HUMAN/253-285 | SDLPAGWMRVQDTSG.-TYYWHIPTGTTQWEPPGR | 3 | 54 |
| APBB1_MOUSE/253-285 | SDLPAGWMRVQDTSG.-TYYWHIPTGTTQWEPPGR | 3 | 55 |
| APBB1_RAT/254-286 | SDLPAGWMRVQDTSG.-TYYWHIPTGTTQWEPPGR | 3 | 56 |
| APBB2_HUMAN/290-322 | PDLPPGWKRVSDIAG.-TYYWHIPTGTTQWERPVS | 3 | 57 |
| APBB2_MOUSE/290-322 | PDLPPGWKRVNDIAG.-TYYWHIPTGTTQWERPVS | 3 | 58 |
| BAG3_HUMAN/20-54 | DPLPPGWEIKIDPQTgWPFFVDHNSRTTTWNDPRV | 3 | 59 |
| BAG3_MOUSE/22-56 | DPLPPGWEIKIDPQTgWPFFVDHNSRTTTWNDPRV | 3 | 60 |
| C1716_DROME/1963- | DPLPPAWNWQVTSDG.DIYYYNLRERISQWEPPSP | 3 | 61 |
| 1996 | | | |
| DMD_CAEEL/3047-3081 | QSVTLPWQRAISKSN1LPYYIEQTSEKTQWEHPVW | 3 | 62 |
| FNBP4_HUMAN/595-629 | NATPKGWSCHWDRDHrRYFYVNEQSGESQWEFPDG | 3 | 63 |
| FNBP4_MOUSE/603-637 | NATPKGWSCHWDRDHrRYFYVNEQSGESQWEFPDG | 3 | 64 |
| IQGA1_HUMAN/679-712 | GDNNSKWVKHWVKGG.YYYYHNLETQEGGWDEPPN | 3 | 65 |
| PCIF1_HUMAN/43-77 | ELVHAGWEKCWSRREnRPYYFNRFTNQSLWEMPVL | 3 | 66 |
| PCIF1_MOUSE/43-77 | ELVHAGWEKCWSRREsRPYYFNRFTNQSLWEMPVL | 3 | 67 |
| PQBP1_BOVIN/46-80 | EGLPPSWYKVFDPSCgLPYYWNVDTDLVSWLSPHD | 3 | 68 |
| PQBP1_GORGO/46-80 | EGLPPSWYKVFDPSCgLPYYWNADTDLVSWLSPHD | 3 | 69 |
| PQBP1_HUMAN/46-80 | EGLPPSWYKVFDPSCgLPYYWNADTDLVSWLSPHD | 3 | 70 |
| PQBP1_MOUSE/46-80 | EGLPPSWYKVFDPSCgLPYYWNVETDLVSWLSPHD | 3 | 71 |
| PQBP1_PONPY/46-80 | EGLPPSWYKVFDPSCgLPYYWNADTDLVSWLSPHD | 3 | 72 |
| PQBP1_RAT/46-80 | EGLPPSWYKVFDPSCgLPYYWNVETDLVSWLSPHD | 3 | 73 |
| PRP40_SCHPO/77-104 | WKEYATADG.KKYWYNVNTRESVWDIPDE | 3 | 74 |
| RHG12_HUMAN/265-298 | IQINGEWETHKDSSG.RCYYYNRGTQERTWKPPRW | 3 | 75 |
| RHG12_MACFA/265-298 | IQINGEWETHKDSSG.RCYYYDRGTQERTWKPPRW | 3 | 76 |
| RHG12_MOUSE/263-296 | IQVNGEWETHKDSSG.RCYYYNRTTQERTWKPPRW | 3 | 77 |
| RHG27_HUMAN/299-333 | VSLETEWGQYWDEESrRVFFYNPLTGETAWEDEAE | 3 | 78 |
| RHG27_MOUSE/299-333 | ESLETEWGQYWDEESrRVFFYNPLTGETAWEDETE | 3 | 79 |
| RHG27_RAT/299-333 | ESLETEWGQYWDEESgRVFFYNPLTGETVWEDETE | 3 | 80 |
| RHG39_HUMAN/63-97 | RTSENQWWELFDPNTsRFYYYNASTQRTVWHRPQG | 3 | 81 |
| RHG39_MOUSE/63-97 | RTSEDQWWELFDPNTsRFYYYSAASQRTVWHRPQN | 3 | 82 |
| SAV1_HUMAN/199-232 | LPLPPGWSVDWTMRG.RKYYIDHNTNTTHWSHPLE | 3 | 83 |
| SAV1_MOUSE/200-233 | LPLPPGWSVDWTMRG.RKYYIDHNTNTTHWSHPLE | 3 | 84 |
| SAV1_RAT/201-234 | LPLPPGWSVDWTMRG.RKYYIDHNTNTTHWSHPLE | 3 | 85 |
| SEC24_CRYNE/5-39 | IMLPQGWEARWDPQAnAYIYVDQSTGRSQWEVPLN | 3 | 86 |
| SEC24_USTMA/6-40 | PQLPPGWVAQWDPNAaRQVFVETATGRTSWQPPTA | 3 | 87 |
| SETD2_HUMAN/2389- | IVLPPNWKTARDPEG.KIYYYHVITRQTQWDPPTW | 3 | 88 |
| 2422 | | | |
| TCRG1_HUMAN/532-561 | TPWCVVWTGDE.RVFFYNPTTRLSMWDRPDD | 3 | 89 |
| TCRG1_MOUSE/534-563 | TPWCVVWTGDE.RVFFYNPTTRLSMWDRPDD | 3 | 90 |
| TCRGL_HUMAN/343-372 | SPWCVVWTGDD.RVFFFNPTMHLSVWEKPMD | 3 | 91 |
| TCRGL_MOUSE/347-376 | SPWCVVWTGDD.RVFFFNPTMQLSVWEKPVD | 3 | 92 |
| WWOX_CHICK/16-49 | EELPPGWEERTTKDG.WVYYANHLEEKTQWEHPKS | 3 | 93 |
| WWOX_DANRE/16-49 | DELPPGWEERSTKDG.WVYYANHEEMKTQWEHPKT | 3 | 94 |
| WWOX_HUMAN/16-49 | DELPPGWEERTTKDG.WVYYANHTEEKTQWEHPKT | 3 | 95 |
| WWOX_MOUSE/16-49 | DELPPGWEERTTKDG.WVYYANHTEEKTQWEHPKT | 3 | 96 |
| WWOX_PONAB/16-49 | DELPPGWEERTTKDG.WVYYANHTEEKTQWEHPKT | 3 | 97 |
| YFB0_YEAST/9-43 | PQVPSGWKAVFDDEYqTWYYVDLSTNSSQWEPPRG | 3 | 98 |
| RH14_ARATH/17-51 | HTLPKPWKGLIDDRTgYLYFWNPETNVTQYEKPTP | 2 | 99 |
| RH14_ORYSJ/21-55 | PTLPKPWRGLIDGNTgYLYFWNPETKAVQYDRPTA | 2 | 100 |
| RH40_ARATH/20-54 | PTLPQPWKGLIDGSTgILYYWNPETNVTQYERPSA | 2 | 101 |
| RH40_ORYSJ/17-51 | PSLPKPWRGLVDGTTgYLYYWNPETNITQYEKPLP | 2 | 102 |
| RH46_ARATH/15-49 | PNLPKPWKGLVDSRTgYLYFWNPETNVTQYERPAS | 2 | 103 |

By “derived from” it is meant that the peptide concerned includes one or more mutations in comparison to the primary amino acid sequence of the peptide on which it was based. Thus a modified WW domain of the invention is considered to be derived from a wild-type protein/peptide sequence, such as from Pin1. Similarly, by “derivative” of a modified WW domain peptide it is meant a peptide sequence that has the selected desired activity (e.g. binding affinity for a selected target ligand), but that further includes one or more mutations or modifications to the primary amino acid sequence of a modified WW domain first identified by the methods of the invention. Thus, a derivative of a modified WW domain of the invention may have one or more (e.g. 1, 2, 3, 4, 5 or more) chemically modified amino acid side chains compared to the modified WW domain from which it is derived. Suitable modifications may include pegylation, sialylation and glycosylation. These may be incorporated through non-natural amino acids or through chemical modification of the natural sequence. In addition or alternatively, a derivative of a modified WW domain may contain one or more (e.g. 1, 2, 3, 4, 5 or more) amino acid mutations, substitutions or deletions to the primary sequence of a selected modified WW domain peptide. Accordingly, the invention encompasses the results of maturation experiments conducted on a modified WW domain to improve or alter one or more characteristics of the initially identified peptide. By way of example, one or more amino acid residues of a selected modified WW domain peptide sequence may be randomly or specifically mutated (or substituted) using procedures known in the art (e.g. by modifying the encoding DNA or RNA sequence). The resultant library or population of derivatised peptides may be selected—by any known method in the art—according to predetermined requirements: such as improved specificity against particular target ligands; or improved drug properties (e.g. stability, solubility, bioavailability, immunogencity etc.). Peptides selected to exhibit such additional or improved characteristics and that display the activity for which the modified WW domain was initially selected may be considered to be derivatives of the modified WW domain and fall within the scope of the invention.

In some cases it may be desirable to conjugate a modified WW domain peptide of the invention to one or more modified WW domains in order to create a multimer, such as a dimer, of modified domains—for example, to bind more than one target sequence simultaneously. The target sequences may be either on the same or different molecules and may be the same or different sequence, depending on requirements.

In some cases it may be desirable to conjugate a modified WW domain peptide of the invention to a non-WW domain moiety. The term “conjugate” is used in its broadest sense to encompass all methods of attachment or joining that are known in the art. For example, the non-WW domain moiety can be an amino acid extension of the C- or N-terminus of the modified WW domain. This amino acid extension may be either a random peptide sequence or a known peptide sequence. In addition, a short amino acid linker sequence may lie between the modified WW domain and the non-WW domain moiety. The invention further provides for molecules where the modified WW domain will be linked, e.g. by chemical conjugation to the non-WW domain moiety optionally via a linker sequence. Typically, the modified WW domain will be linked to the other moiety via sites that do not interfere with the activity of either moiety. The term “conjugated” is used interchangeably with the terms such as “linked”, “bound”, “associated” or “attached”. A wide range of covalent and non-covalent forms of conjugation are known to the person of skill in the art, and fall within the scope of the invention. For example, disulphide bonds, chemical linkages and peptide chains are all forms of covalent linkages. Where a non-covalent means of conjugation is preferred, the means of attachment may be, for example, a biotin-(strept)avidin link or the like. Antibody (or antibody fragment)-antigen interactions may also be suitably employed to conjugate a modified WW domain of the invention to a non-WW domain moiety. One suitable antibody-antigen pairing is the fluorescein-antifluorescein interaction.

A “non-WW domain moiety” as used herein, refers to an entity that does not contain a WW domain framework or fold. Thus, it does not include a three-stranded β-sheet or β-meander. Such non-WW domain moieties include nucleic acids and other polymers, peptides, proteins, peptide nucleic acids (PNAs), antibodies, antibody fragments, and small molecules, amongst others. Advantageously, a non-WW domain moiety may be a therapeutic or targeting molecule.

By “non-target” in it meant that the ligand concerned is not bound by the relevant WW domain peptide. For example, where a wild-type WW domain has a single specific known natural target ligand, all other peptide sequences would be non-target. A non-target ligand is not bound by the relevant WW domain. By “non-binding”, “not bound” or “not recognised” and equivalent statements it is meant that the ligand concerned is not appreciable bound such that any binding is sub-physiological (i.e. not capable of creating a physiological response under physiological ligand/domain concentrations). For example, if any binding can be measured between the domain and the non-target ligand, the dissociation constant (Kd) is greater than 1 μM, such as at least 100 μM, or at least 1 mM). However, recognising that the binding affinity of different wild-type domains for natural target ligands can significantly vary, in another way, non-binding can be judged relative to target ligand binding. Conveniently, therefore, the dissociation constant for the domain and its target ligand (whether natural in the case of a wild-type WW domain, or natural or non-natural in the case of a modified WW domain) is at least 10-fold higher than for the non-target ligand, suitably at least 100-fold higher, and more suitably at least 1000-fold higher.

The term “extracellular” in the context of a ligand for the WW domains of the invention applies to a molecule that in its natural state can be found in an extracellular environment in vivo, or to a portion of a molecule that is found in an extracellular environment in vivo. For example, the extracellular domain of a membrane-bound or transmembrane molecule (e.g. a membrane-bound growth factor or hormone receptor molecule or complex) may provide an extracellular ligand for a WW domains of the invention.

### WW Domains, Frameworks and Libraries

In the present invention, we have created a WW domain framework suitable for the generation of libraries of modified WW domain peptides, which can be screened for desirable properties, such as binding affinity to a chosen target ligand.

There are a number of WW domains known in the art, and any of these WW domains may be suitable for use as WW domain frameworks for the selection of novel binding modules, as described herein. Thus, suitable WW domains for use in accordance with the invention include polypeptides comprising the WW domain sequences identified in Table 2. Preferably, suitable WW domain sequences include those domains with at least 3 invariant tryptophan residues within the sequence, whether as part of the wild-type sequence or introduced artificially.

In one embodiment the WW domain framework is based on the wild-type Pin1 WW domain peptide sequence, i.e. N′-MADEEKLPPGWEKRMSRSSGRVYYFNHITNASQWERPSG-C′ (SEQ ID NO: 1), which is a group IV WW domain that has only been demonstrated to bind linear peptide sequences containing phosphoserine and/or phosphothreonine residues. The wild-type Pin1 WW domain is thus considered to be a 39 amino acid peptide sequence, with an N-terminal methionine residue at position 1 and a C-terminal glycine residue is at position 39. However, it should be appreciated that the N-terminal Met-Ala dipeptide may be optionally omitted from the WW domain sequence and, therefore, WW domain frameworks and modified peptide and nucleic acid sequences omitting the N-terminal Met-Ala dipeptide of the wild-type sequence are also encompassed within the scope of the invention and are considered disclosed herein. For ease of understanding the invention and for reason of internal consistency, as used herein, the numbering of amino acid positions in Pin1 WW domains, WW domain frameworks and modified WW domain peptides of the invention that have been derived from Pin1 will be in accordance with the numbering of SEQ ID NO: 1 (which includes methionine at position 1, alanine at position 2 and glycine at position 39), unless otherwise stated. Thus, where an amino acid position is given herein with reference to a specific sequence, such as SEQ ID NOs: 15 to 20 or 26 to 29, the numbering of the specific sequence applies; otherwise, the numbering is assumed to be with reference to SEQ ID NO: 1.

In one embodiment the WW domain framework of the invention has an amino acid deletion in the turn encompassing positions 17 (Arg) to 20 (Gly), i.e. the “loop 1 region”, so that the framework includes 38 residues. This deletion creates a one amino acid truncation in the loop1 region of the Pin1 WW domain, which may help to thermally stabilise the domain. In another embodiment Met15 of the Pin1 WW domain of SEQ ID NO: 1 is mutated to a tryptophan (M15W), which may also assist in stabilising the WW domain. Suitably, the WW domain framework of the invention comprises both the deletion of an amino acid between positions 17 and 20 and the mutation M15W in comparison to the Pin1 sequence, so as to create a WW domain framework of increased stability in comparison to the wild-type Pin1 WW domain, such as SEQ ID NO: 2. A preferred deletion in the loop 1 region is the deletion of Ser19 in SEQ ID NO: 1 (or equivalent position when the WW domain is derived from an alternative source). This one amino acid deletion means that the sequences of SEQ ID NOs: 15 to 20 and 26 to 29, for example, are at least one amino acid shorter than that of SEQ ID NO: 1 and means that Ser38 of SEQ ID NO: 1 corresponds to position 37 of SEQ ID NOs: 15 to 20 and 26 to 29.

In another embodiment the WW domain framework of the invention has an insertion of at least one amino acid in the turn encompassing positions 17 to 20 (loop 1) and/or in the turn encompassing positions 27 to 30 (i.e. the loop 2 region), so that the framework may include more than 39 residues. These insertions create extended loop regions that may be used to provide further diversity and an increased number of amino acid side chains for ligand recognition. Preferably a stabilised loop sequence is used so that the extended loop has structural stability and does not inhibit or destabilise the folding of the three-stranded beta-sheet structure of a WW domain. In some embodiments a loop sequence, such as a hyper-variable loop region from an antibody (or antibody fragment), may be grafted onto the WW domain of the invention. 1 or more amino acids may be inserted into the loop 1 and/or loop 2 regions, for example, up to 10, up to 8, or up to 5 amino acids. In naïve WW domain libraries of the invention the inserted amino acids may be subject to diversification (i.e. they may be part of the variable library residues), in order that specific, useful sequences can be selected from the library in the methods of the invention. Alternatively, one or more of the amino acids of the insertion may be invariable, e.g. for reason of structural stability or side chain properties.

In yet another embodiment the WW domain framework comprises at least one mutation at a position selected from positions 12, 14, 17, 18, 23, 25, 27, 30 and 32 of the wild-type WW domain sequence. When the WW domain framework is derived from Pin1, the one or more mutations are therefore selected from positions E12, R14, R17, S18, Y23, F25, H27, N30 and S32. These positions are spread over both loops (loop 1 and loop 2) of the WW domain, as well as over the putative binding/recognition surface of the β-meander (see FIG. 1). Suitably, the framework includes amino acid substitutions of at least 3, at least 5 or at least 7 of positions 12, 14, 17, 18, 23, 25, 27, 30 and 32 of SEQ ID NO: 1. In more suitable embodiments the framework of the invention includes amino acid substitutions at 8 or 9 of the above-identified positions of SEQ ID NO: 1. Advantageously, in addition to the above mutations, the WW domain contains at least 3 tryptophan residues (as highlighted in Table 2 above and Table 3).

The amino acid residues at each of the mutated positions may be non-selectively randomised, i.e. by replacing each of the specified amino acids with one of the other 19 naturally occurring amino acids; or may be selectively randomised, i.e. by replacing each of the specified amino acids with one from a defined sub-group of the remaining 19 naturally occurring amino acids. The mutations may also encompass non-natural amino acids. It will be appreciated that one convenient way of creating a library of mutant peptides with randomised amino acids at each selected location, is to randomise the nucleic acid codon of the corresponding nucleic acid sequence that encodes the selected amino acid. In this case, in any individual peptide expressed from the library, any of the 20 naturally occurring amino acids may be incorporated at the randomised position. Therefore, in some instances (e.g. approximately 5%), the wild-type amino acid residue may be ‘randomly’ incorporated by chance. Therefore, while a WW domain framework nucleic acid of the invention may have randomised codons in all 9 of the above-identified positions, a modified WW domain peptide may have a wild-type amino acid in one or more of those selected positions. In contrast, by substituting a selected amino acid of the wild-type sequence with one from a defined sub-group of amino acids (e.g. by intelligent codon randomisation), it can be pre-determined whether or not any of the library members might incorporate a wild-type residue at the selected location by chance. Alternatively precharged tRNAs may be used to introduce non-natural amino acids at any one or more of the positions to be mutated. Other methods of tRNA aminoacylation with non-natural amino acids include the use of ribozymes or mutated aminoacyl-tRNA synthetases (AARS) which may have specific four base codons (Ullman et al., 2011, Briefings in Functional Genomics, 10, 125-134).

In one embodiment of the WW domain framework, positions E12, R14, Y23, F25, H27 and N30 are substituted for any one of the 20 naturally-occurring amino acids, and positions R17, S18 and S32 are substituted for any one of the amino acids of the group A, G, N, K, D, E, R, T, S, P, H and Q. More suitably, the WW domain framework further comprises one or both of the deletion of Ser19 and the substitution Met15Trp.

Thus, a naïve WW domain framework peptide library of the invention may have the sequence:

N′-MADEEKLPPGWX₁KX₂WSX₃X₄GRVX₅YX₆NX₇ITX₈AX₉QWERPSG-C′ (SEQ ID NO: 2); wherein X₁, X₂, X₅, X₆, X₇, X₈ represent any one of the 20 naturally-occurring amino acids, and X₃, X₄ and X₉ represent any one of amino acids A, G, N, K, D, E, R, T, S, P, H and Q; Met15 of the corresponding wild-type Pin1 sequence has been replaced with Trp and Ser19 of the wild-type Pin1 WW domain has been deleted. In the corresponding WW domain framework nucleic acid library (termed PinLib, SEQ ID NO: 3): X₁, X₂, X₅, X₆, X₇, X₈ were encoded by an NNB codon, wherein N represents an equal mixture of A, C, T and G, and B is C, G, and T; and X₃, X₄ and X₉ were encoded by a VVM codon, wherein V is A, C or G, and M is A or C. It should be appreciated, however, that the end regions of such peptides may be amenable to modifications and deletions or additions without adversely affecting the folding or binding specificity of the WW domain of the invention (e.g. the N and C-terminal amino acids are not thought to be directly involved in binding interactions with a target ligand). Hence, a preferred WW domain framework library for the selection of WW domain peptides, and thus, useful WW domain peptides of the invention may comprise the amino acid sequence: KLPPGWX₁KX₂WSX₃X₄XₐGRVX₅YX₆NX₇ITX₈AX₉QWERP where X₁ to X₉ represent any amino acid and Xₐ is optionally any amino acid or absent (SEQ ID NO: 31), which corresponds either to the sequence of amino acids 6 to 38 of a modified SEQ ID NO: 1 sequence, or amino acids 6 to 37 of SEQ ID NO: 2. A most preferred WW domain framework library and most preferred WW domain peptides of the invention may, therefore, comprise the amino acid sequence: KLPPGWX₁KX₂WSX₃X₄GRVX₅YX₆NX₇ITX₈AX₉QWERP where X₁ to X₉ represent any amino acid (SEQ ID NO: 32), or may be defined as indicated above. Depending on the target ligand, preferred sub-groups of amino acids for incorporation at each X position (as for SEQ ID No: 2) are reported in the Examples; and are incorporated within the scope of the invention.

In some cases it may be beneficial to express WW domain framework peptides as a fusion protein, for example, to aid in the expression, screening or selection of desirable modified WW domain peptides. In one embodiment, the WW domain framework peptides are expressed with a linker sequence at the N- or C-terminus. In a particularly preferred embodiment the WW domain framework peptides include a GSGSS (SEQ ID NO: 33) amino acid linker at the C-terminus and the WW domain framework nucleic acid sequence includes a corresponding nucleic acid sequence. This linker is convenient for fusing WW domain framework peptides of the invention to the RepA protein for expression and selection in a CIS in vitro display system.

While the above has been described primarily in relation to a WW domain framework derived from Pin1, it will be appreciated that other WW domains may alternatively be used, such as those listed in Tables 2 or 3. In some cases it may be desirable to use a framework derived from a particular animal or organism, such as a human, primate, porcine or murine. In other cases it may be beneficial to derive a framework from a hybrid WW domain: for example, one or more loop sequences or β-strands of one WW domain may be replaced by the loop or β-strand sequences of a different WW domain to provide a different (non-natural) combination of properties. Particularly suitable WW domains from which a WW domain framework of the invention may be derived include those listed in Table 2, in particular formin binding protein 4 (FNBP4), Amyloid beta A4 precursor protein-binding family B member 1, 2 or 3 (APBB1, 2, 3), PCIF1, Polyglutamine-binding protein 1 (PQBP1), PRP40, RHG12, RHG27, Protein salvador homolog 1 (SAV1), BCL2-associated athanogene product (BAG3), SEC23, T-cell receptor gamma protein (TCRG), WWC, WWOX or YFB protein (Table 3).

TABLE 3
Position of Trp residues in selected natural WW domain sequences
shown in Table 2, which contain at least 3 Trp residues,
and comparison with the invariable Trp positions
in a naïve Pin library according to the invention.

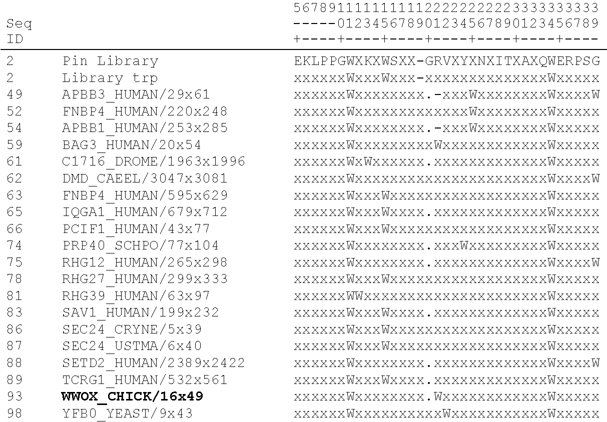

‘Pin Library’ sequence is based on SEQ ID NO: 2.
The positions of the Trp residues in the sequences are shown as W while all other
amino acids are indicated by x.
The complete amino acid sequences corresponding to the respective SEQ ID NOs
are shown in Table 2.

Expression and Characterisation of Peptides from Libraries

The modified WW domain peptides of the invention may conveniently be selected by screening libraries of peptides derived from a WW domain framework. The screening may be performed using any library generation and selection system known to the person of skill in the art, such as those identified below.

One approach is to produce a mixed population of candidate peptides by chemically synthesising a randomised library of 6 to 10 amino acid peptides (J. Eichler et al., (1995) Med. Res. Rev., 15, 481-496; K. Lam (1996) Anticancer Drug Des., 12, 145-167; and M. Lebl et al., (1997) Methods Enzymol., 289, 336-392). In another approach, candidate peptides are synthesised by cloning a randomised oligonucleotide library into an Ff filamentous phage gene, which allows peptides that are much larger in size to be expressed on the surface of the bacteriophage (H. Lowman (1997) Ann. Rev. Biophys. Biomol. Struct., 26, 401-424; and G. Smith et al., (1993) Meth. Enz., 217, 228-257). Randomised peptide libraries up to 38 amino acids in length have also been made, and longer peptides are achievable using this system. The peptide libraries that are produced using either of these strategies are then typically mixed with a pre-selected matrix-bound protein target. Peptides that bind are eluted, and their sequences are determined. From this information new peptides are synthesised and their biological properties can be assessed.

A potential disadvantage of such prior art procedures is that the size of the libraries that are typically generated with both phage display and chemical synthesis is limited to within the 10⁶-10⁹ range. This limitation can result in the isolation of peptides of relatively low binding affinity for the target ligand, unless a time-consuming maturation process is subsequently used. This library-size limitation has led to the development of techniques for the in vitro generation of peptide libraries including: mRNA display (Roberts, & Szostak (1997) Proc. Natl. Acad. Sci. USA, 94, 12297-12302); ribosome display (Mattheakis et al., (1994) Proc. Natl. Acad. Sci. USA, 91, 9022-9026); and CIS display (Odegrip et al., (2004) Proc. Natl. Acad. Sci. USA, 101 2806-2810) amongst others. These libraries can be superior to phage display libraries (and other in vivo-based procedures), in that the size of libraries generated may be 2-5 orders of magnitude larger than is possible with phage display. This is because unlike techniques such as phage display, there are no intermediate in vivo steps requiring the cloning and transformation of nucleic acid sequences from the library. Therefore, the expression and selection of library members may be beneficially performed using in vitro display of in vitro generated libraries of peptides derived from a WW domain framework.

The terms “in vitro display”, “in vitro peptide display” and “in vitro generated libraries” as used herein refer to systems in which peptide libraries are expressed in such a way that the expressed peptides associate with the specific nucleic acids that encoded them, and the association does not follow or require the transformation of cells or bacteria with the nucleic acids. Accordingly, these systems can be considered to be “acellular”. Such systems contrast with phage display and other “cellular” or “in vivo display” systems in which the association of peptides with their encoded nucleic acids follows the transformation of cells or bacteria with the nucleic acids. In a preferred embodiment of the invention, the CIS-display system (for example, as described in WO2004022746, WO2006097748 and WO2007010293) is used as an in vitro display system for the selection of modified WW domains.

The binding affinity of a selected modified WW domain peptide for the selected target ligand can be measured using techniques known to the person of skill in the art, such as tryptophan fluorescence emission spectroscopy, isothermal calorimetry, surface plasmon resonance, or biolayer interferometry. Biosensor approaches are reviewed by Rich et al. (2009), “A global benchmark study using affinity-based biosensors”, Anal. Biochem., 386, 194-216. For example, in one method the dissociation constant for a WW domain is determined by titration of the domain against a ligand in a suitable buffer solution, such as 10 mM potassium phosphate, 100 mM NaCl, 0.1 mM EDTA, 0.1 mM DTT (pH 6.0). The domain and ligand are mixed for 2 minutes and the change in tryptophan fluorescence is monitored following excitation at 298 nM and detection at 340 or 350 nM. The data points are typically averaged over a 10 second period. Alternatively, real-time binding assays between a chemically synthesised WW domain and ligand may be performed using biolayer interferometry with an Octet Red system (Fortebio, Menlo Park, Calif.). Biotinylated WW domain is immobilised to streptavidin biosensors (Fortebio) at a concentration of 50 ng/μl in PBS. Association curves are then detected by incubating WW domain-coated sensors with different concentrations of ligand (e.g. 1 to 100 nM in PBS), and dissociations detected by incubating the sensors in PBS. The dissociation constant for WW domain binding can be determined by steady state analysis.

Modified WW domain peptides of the invention have pM or higher binding affinity for a target ligand. Suitably, a modified WW domain peptide of the invention has nM or sub-nM binding affinity for its target ligand; for example, less than 1 μM, less than approximately 900 nM; less than approximately 700 nM, less than approximately 500 nM or less than approximately 300 nM. In still more suitable embodiments, the modified WW domain peptide has an affinity (as measured by a suitable dissociation constant) of less than approximately 200 nM, less than approximately 100 nM or less than approximately 10 nM. In some particularly preferred embodiments the affinity of the modified WW domain peptide for its target ligand is in the pM range.

Preferably, the target ligand is a ligand that is a non-natural ligand of the wild-type WW domain from which the modified WW domain peptide is derived. In such embodiments, the affinity of the modified WW domain peptide for its (non-natural) target ligand is suitably at least 10-fold greater than it is for the natural ligand of the corresponding wild-type WW domain, which can then be considered a non-target ligand for the modified domain. More suitably, the affinity for the (non-natural) target ligand is at least 50-fold, at least 100-fold, or at least 1000-fold greater than it is for the natural ligand of the corresponding wild-type WW domain.

In some embodiments, the target ligand for the modified WW domain may be the same as the natural target ligand of the corresponding wild-type WW domain. In these embodiments the affinity of the modified WW domain peptide for the target ligand is beneficially at least 2-fold higher or at least 5-fold higher than that of the wild-type WW domain. Suitably, so that the increase in affinity of the modified domain for the natural ligand is readily apparent, the increase in affinity over the wild-type peptide sequence is at least 10-fold, at least 25-fold or at least 50-fold higher than that of the wild-type domain. In some particularly advantageous embodiments, the affinity of the modified WW domain peptide for the target ligand is at least 100-fold or at least 1000-fold higher than that of the wild-type WW domain.

Screening and Selection of Peptides from Libraries

The present invention represents a significant advance in the art of peptide scaffolds/frameworks for the generation and selection of peptides having desirable properties from libraries (e.g. naïve libraries), and also in drug development by allowing screening of peptide libraries for desirable pharmaceutical properties.

In accordance with one embodiment of this aspect of the invention, in vitro generated nucleic acid libraries encoding a plurality of modified WW domain peptides are synthesised and initially selected for their ability to bind a desired target ligand. In a particularly advantageous method the peptides are synthesised in a CIS in vitro display system, in which each modified WW domain peptide is expressed as a fusion protein to RepA, which binds a target sequence in the nucleic acid (DNA) molecule that encodes the fusion protein, thus forming a complex. In this way, the modified WW domain (peptide) is linked to the nucleic acid that encoded it (i.e. genotype and phenotype are linked), as a peptide-nucleic acid complex.

The ligand may be a naturally or non-naturally occurring molecule, such as an organic or inorganic small molecule, a carbohydrate, peptide or protein sequence. It may be a whole molecule or a part of a larger molecule (e.g. a domain, fragment or epitope of a protein), and may be an intracellular or an extracellular target molecule. In a beneficial embodiment the target is an extracellular ligand, which may be more readily targeted for therapeutic uses.

Conveniently, to aid in the separation of ligand-bound modified WW domain peptides from free WW domains, the ligands may be associated with or otherwise attached to a solid support. By way of example, the solid support may be the surface of a plate, tube or well; alternatively the solid support may be a bead, such as a magnetic or agarose bead. In one example, the bead is a polystyrene-coated magnetic bead. The solid support may be coated with the ligand using any appropriate method. For instance, a ligand may be added to magnetic beads, for example, TALON® magnetic beads (Invitrogen, USA), in suitable buffer (such as PBS) and incubated for a period of time. The incubation can conveniently be carried out at room temperature whilst mixing on a rotary mixer. Before use the beads may be washed, for example, 3 times with PBS buffer.

The ligand (preferably immobilised) is then contacted with the library of WW domain framework peptides, typically by incubating the expressed WW domain peptides with the ligand. In one convenient embodiment, the peptide library is expressed in an in vitro combined transcription and translation system, which does not require the use of cloning and transformation steps. After a suitable incubation time, magnetic beads may be pelleted under gravity and/or magnetic force, for example, so as to separate ligand-bound peptide-nucleic acid complexes from non-associated complexes which remain in free solution/suspension.

Non-associated complexes may be removed by aspiration and, typically, with one or more washing steps using suitable buffers and/or detergents; or by any other means known to the person of skill in the art. A convenient buffer is PBS, but other suitable buffers known in the art may also be used. By washing the mixture, library members that are incapable of associating with the target ligand (or which associate too weakly to remain associated under washing conditions), and free nucleic acid molecules (e.g. from dissociated complexes) can be removed from the selection.

At least one round of expression, binding and selection is performed in order to enrich the population of modified WW domain peptides (and their associated nucleic acids) for the desired characteristic(s). Typically, 2, 3, 4, 5 or more rounds of selection may be carried out. In each subsequent round of selection certain criteria, particularly binding conditions, may be modified, for example, to enhance the selection of modified WW domain peptides having desirable properties, such as high affinity, increased specificity and so on.

At the end of each round of selection and at the end of the procedure, the ligand-associated modified WW domain peptides may then be recovered and individually characterised by sequencing the associated nucleic acid. Optionally, the peptides may be further characterised by expressing or synthesising the encoded WW domain to confirm the desired ligand-binding properties. Advantageously, the modified WW domain peptides and/or nucleic acids of the invention may be isolated. However, a mixed population of modified WW domains may be obtained by the methods of the invention, e.g. where more than one peptide-nucleic acid complex associates with the target ligand. In this event, the invention also encompasses a mixed population of modified WW domains that bind a target ligand.

In a modification of the above method, where it is intended for the modified WW domain peptides to have a therapeutic use in an animal, such as a human, the modified WW domain peptides may be exposed to one or more protease(s) that are endogenous to the target animal (e.g. those in the circulating blood flow of the animal concerned), in order to further select for resistance to a particular protease. By way of example, the proteases may be selected from one or more human protease, e.g. chymotrypsin, trypsin, aminopeptidases, carboxypeptidases and elastases.

The selection and screening methods of the invention can be applied to the selection of modified WW domain peptides for binding to any desired target ligand. Suitable ligands may include growth factors, receptors, channels, abundant serum proteins, hormones, microbial antigens. Specific examples of potential target ligands include VEGFR2 and NGF.

### Nucleic Acids and Peptides

The modified WW domain peptides according to the invention and, where appropriate, the modified WW domain peptides conjugated to non-WW domain peptide moieties may be produced by recombinant DNA technology and standard protein expression and purification procedures. Thus, the invention further provides nucleic acid molecules that encode the WW domain peptides of the invention as well as their derivatives, and nucleic acid constructs, such as expression vectors that comprise nucleic acids encoding peptides and derivatives according to the invention. For instance, the DNA encoding the relevant peptide can be inserted into a suitable expression vector (e.g. pGEM®, Promega Corp., USA), where it is operably linked to appropriate expression sequences, and transformed into a suitable host cell for protein expression according to conventional techniques (Sambrook J. et al., Molecular Cloning: a Laboratory Manual, Cold Spring Harbor Press, Cold Spring Harbor, N.Y.). Suitable host cells are those that can be grown in culture and are amenable to transformation with exogenous DNA, including bacteria, fungal cells and cells of higher eukaryotic origin, preferably mammalian cells. To aid in purifying the peptides of the invention, the WW domain peptide (and corresponding nucleic acid) of the invention may include a purification sequence, such as a His-tag. In addition, or alternatively, the modified WW domain peptides may, for example, be grown in fusion with another protein and purified as insoluble inclusion bodies from bacterial cells. This is particularly convenient when the modified WW domain peptide to be synthesised may be toxic to the host cell in which it is to be expressed. Alternatively, modified WW domain peptides may be synthesised in vitro using a suitable in vitro (transcription and) translation system (e.g. the E. coli S30 extract system, Promega corp., USA).

The term “operably linked”, when applied to DNA sequences, for example in an expression vector or construct indicates that the sequences are arranged so that they function cooperatively in order to achieve their intended purposes, i.e. a promoter sequence allows for initiation of transcription that proceeds through a linked coding sequence as far as the termination sequence.

Having selected and isolated a desired modified WW domain peptide, an additional functional group such as a second therapeutic molecule may then be attached to the WW domain peptide by any suitable means. For example, a modified WW domain peptide may be conjugated to any suitable form of therapeutic molecule, such has an antibody, enzyme or small chemical compound. This can be particularly useful in applications where the modified WW domain peptide of the invention is capable of targeting or associating with a particular cell or organism that can be treated by the second therapeutic molecule. A preferred form of therapeutic molecule that may be attached or linked to a peptide or nucleic acid of the invention is an siRNA molecule capable of inducing RNAi in a target cell. Typically a chemical linker will be used to link an siRNA molecule to a peptide, such as a WW domain. For example, the nucleic acid or PNA can be linked to the WW domain peptide through a maleimide-thiol linkage, with the maleimide group being on the peptide and the thiol on the nucleic acid, or a disulphide link with a free cysteine group on the peptide and a thiol group on the nucleic acid. WW domain peptides may also be conjugated to a molecule that recruits immune cells of the host. Such conjugated WW domain peptides may be particularly useful for use as cancer therapeutics.

In a further alternative, the WW domain may be directly conjugated to an antibody molecule, an antibody fragment (e.g. Fab, F(ab)₂, scFv etc.) or other suitable targeting agent, so that the modified WW domain peptide and any additional conjugated moieties are targeted to the specific cell population required for the desired treatment or diagnosis—for example, producing a bi-functional binder so that two separate cells can be targeted by the same WW domain-antibody fusion or two separate receptors can be bound by the WW domain-antibody fusion.

### Therapeutic Compositions

A modified WW domain peptide of the invention may be incorporated into a pharmaceutical composition for use in treating an animal; preferably a human. A therapeutic peptide of the invention (or derivative thereof) may be used to treat one or more diseases or infections, dependent on what ligand to select modified WW domain peptides from a WW domain framework library. Alternatively, a nucleic acid encoding the therapeutic peptide may be inserted into an expression construct and incorporated into pharmaceutical formulations/medicaments for the same purpose.

The therapeutic peptides of the invention may be particularly suitable for the treatment of diseases, conditions and/or infections that can be targeted (and treated) extracellularly, for example, in the circulating blood or lymph of an animal; and also for in vitro and ex vivo applications. Therapeutic nucleic acids of the invention may be particularly suitable for the treatment of diseases, conditions and/or infections that are more preferably targeted (and treated) intracellularly, as well as in vitro and ex vivo applications. As used herein, the terms “therapeutic agent” and “active agent” encompass both peptides and the nucleic acids that encode a therapeutic modified WW domain peptide of the invention.

Therapeutic uses and applications for the modified WW domain peptides and nucleic acids of the invention include: anti-VEGF agents for treatment of various neoplastic and non-neoplastic diseases and disorders; cancers/neoplastic diseases and related conditions (such as breast carcinomas, lung carcinomas, gastric carcinomas, esophageal carcinomas, oral carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema—such as that associated with brain tumors, and Meigs' syndrome); non-neoplastic conditions, such as head injury, spinal cord injury, acute hypertension, meningitis, encephalitis, abscess, haemorrhage, cerebral malaria, radiation, multiple sclerosis, cardiac arrest, birth asphyxia, glutamate toxicity, encephalopathy, hypoxia, ischemia, or renal dialysis, stroke, endometriosis, rheumatoid arthritis, psoriasis, atherosclerosis, diabetic and other proliferative retinopathies including retinopathy of prematurity, diabetes, diabetic macular oedema, retrolental fibroplasia, neovascular glaucoma, age-related macular degeneration, thyroid hyperplasias (including Grave's disease), corneal and other tissue transplantation, chronic inflammation, lung inflammation, nephrotic syndrome, preeclampsia, ascites, pericardial effusion (such as that associated with pericarditis), and pleural effusion and oedema. Other therapeutic uses for the molecules and compositions of the invention include the treatment of microbial infections and associated conditions, for example, bacterial, viral, fungal or parasitic infection.

One or more additional pharmaceutically acceptable carrier (such as diluents, adjuvants, excipients or vehicles) may be combined with the therapeutic peptide of the invention in a pharmaceutical composition. Suitable pharmaceutical carriers are described in “Remington's Pharmaceutical Sciences” by E. W. Martin. Pharmaceutical formulations and compositions of the invention are formulated to conform to regulatory standards and can be administered orally, intravenously, topically, or via other standard routes.

In accordance with the invention, the therapeutic peptide or nucleic acid may be manufactured into medicaments or may be formulated into pharmaceutical compositions. When administered to a subject, a therapeutic agent is suitably administered as a component of a composition that comprises a pharmaceutically acceptable vehicle. The molecules, compounds and compositions of the invention may be administered by any convenient route, for example, intradermal, intramuscular, intraperitoneal, intravenous, subcutaneous, intranasal, epidural, oral, sublingual, intranasal, intravaginal, transdermal, rectally, by inhalation, or topically to the skin. Administration can be systemic or local. Delivery systems that are known also include, for example, encapsulation in microgels, liposomes, microparticles, microcapsules, capsules, etc., and can be used to administer the compounds of the invention. Any other suitable delivery systems known in the art are also envisioned in use of the present invention.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are preferably sterile. Water is a suitable vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the invention can take the form of liquids, solutions, suspensions, lotions, gels, tablets, pills, pellets, powders, modified-release formulations (such as slow or sustained-release), suppositories, emulsions, aerosols, sprays, capsules (for example, capsules containing liquids or powders), liposomes, microparticles or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

Suitably, the therapeutic compositions or medicaments of the invention are formulated in accordance with routine procedures as a pharmaceutical composition adapted for oral administration (more suitably for human beings). Compositions for oral delivery may be in the form of tablets, lozenges, aqueous or oily suspensions, granules, powders, emulsions, capsules, syrups, or elixirs, for example. Thus, in one embodiment, the pharmaceutically acceptable vehicle is a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunem, or the ileum), or the large intestine. One skilled in the art is able to prepare formulations that will not dissolve in the stomach, yet will release the material in the duodenum or elsewhere in the intestine. Suitably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 would be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

To aid dissolution of the therapeutic agent or nucleic acid (or derivative) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the peptide or nucleic acid or derivative either alone or as a mixture in different ratios.

Typically, compositions for intravenous administration comprise sterile isotonic aqueous buffer. Where necessary, the compositions may also include a solubilising agent.

Another suitable route of administration for the therapeutic compositions of the invention is via pulmonary or nasal delivery.

Additives may be included to enhance cellular uptake of the therapeutic peptide (or derivative) or nucleic acid of the invention, such as the fatty acids oleic acid, linoleic acid and linolenic acid.

In one pharmaceutical composition, a modified WW domain peptide or nucleic acid of the invention (and optionally any associated non-WW domain moiety, e.g. a therapeutic molecule or targeting moiety) may be mixed with a population of liposomes (i.e. a lipid vesicle or other artificial membrane-encapsulated compartment), to create a therapeutic population of liposomes that contain the therapeutic agent and optionally the non-WW domain moiety. The therapeutic population of liposomes can then be administered to a patient by any suitable means, such as by intra-venous injection. Where it is necessary for the therapeutic liposome composition to target specifically a particular cell-type, such as a particular microbial species or an infected or abnormal cell, the liposome composition may additionally be formulated with an appropriate antibody domain or the like (e.g. Fab, F(ab)₂, scFv etc.) or alternative targeting moiety, which recognises the target cell-type. Such methods are known to the person of skill in the art.

The therapeutic peptides or nucleic acids of the invention may also be formulated into compositions for topical application to the skin of a subject.

Modified WW domain peptides and nucleic acids of the invention may also be useful in non-pharmaceutical applications, such as in diagnostic tests, imaging, as affinity reagents for purification and as delivery vehicles.

The invention will now be further illustrated by way of the following non-limiting examples.

### EXAMPLES

Unless otherwise indicated, commercially available reagents and standard techniques in molecular biological and biochemistry were used.

### Materials and Methods

The following procedures used by the Applicant are described in Sambrook, J. et al., 1989 supra.: analysis of restriction enzyme digestion products on agarose gels and preparation of phosphate buffered saline. General purpose reagents were purchased from Sigma-Aldrich Ltd (Poole, Dorset, UK). Oligonucleotides were obtained from Sigma Genosys Ltd (Haverhill, Suffolk, UK) or Genelink Inc., (Hawthorne, N.Y., USA). Amino acids, and S30 extracts were obtained from Promega Ltd (Southampton, Hampshire, UK). Enzymes and polymerases were obtained from New England Biolabs (NEB) (Hitchin, UK). Fmoc amino acids were supplied by Activotec (Cambridge, UK), or Merck Chemicals (Darmstadt, Germany), and chemicals and solvents were purchased from Fisher Scientific (Loughborough, Leicestershire, UK).

### Example 1

### A. Library Construction

The modified Pin1 WW domain framework library (for generation of libraries of modified Pin1 WW domains), was created by randomising residues situated on one surface of a mutated Pin1 domain (FIG. 1). SEQ ID NO: 1 shows the amino acid sequence of the wild-type Pin1 WW domain. The peptide framework was constructed in three stages:

(1) Loop 1 of the wild-type sequence was shortened by 1 amino acid;
(2) Met15 was substituted for Trp; and
(3) 9 of the remaining amino acid positions were randomly mutated.

The first two stages generate a specifically mutated Pin1 WW domain framework that has higher thermostability than the wild-type Pin1 WW domain (SEQ ID NO: 12). The third stage then creates a naïve framework library from which modified WW domains having desirable properties can be selected.

The framework peptide library is created by randomised the amino acids at positions X₁-X₉ in SEQ ID NO: 2 using PCR to mutate the corresponding nucleic acid encoding sequence (SEQ ID NO: 3). As illustrated, to produce the randomisations, the nucleotide triplets that encode the amino acids at positions X₁, X₂, X₅, X₆, X₇, X₈ are mutated to NNB (where N represents an equal mixture of A, C, T and G; and B can be C, G, T), to allow any of the 20 naturally occurring amino acids to be incorporated at these position. The nucleotide triplets encoding positions X₃, X₄ and X₉ of SEQ ID NO: 2 were mutated to a VVM codon (where V is A, C, G; and M is A or C), which encodes any of 12 amino acids (i.e. A, G, N, K, D, E, R, T, S, P, H and Q). The selection of this sub-group of amino acids was made in order to increase the relative proportion of structurally functional residues in the library by encoding those amino acid side-chains that were thought most likely to be accommodated in a turn or loop. It will be appreciated that because of the codon usage for different amino acids, the representation of different amino acids at each randomised position may not be equally distributed. In addition, it will be appreciated that other codons may also be accommodated in positions X₃, X₄ and X₉ that may contribute to stability or activity.

To express each of the modified Pin1 WW domain peptides in the CIS display library, the framework nucleic acid library includes a 3′ sequence that encodes a GSGSS (SEQ ID NO: 33) amino acid linker at the C-terminus of the WW domain peptide (SEQ ID NO: 14). The GSGSS sequence links the C-terminus of the WW domain to the N-terminus of the RepA protein.

In vitro (CIS-display) library construction was carried out generally as described by Odegrip et al. (2004, Proc. Natl. Acad. Sci. USA, 101 2806-2810). All enzymes were purchased from New England Biolabs (NEB Ltd, Hitchin, UK). All PCRs contained 200 pmol of each primer, 2.5 unit of Taq DNA polymerase, 200 μM each dNTP (NEB Ltd, Hitchin, UK) and 1× ThermoPol buffer per 50 μl PCR reaction. PCRs were carried out on a Techne Techgene PCR machine (Fisher Scientific, Loughborough, UK) for one cycle of 2 min at 94° C., followed by 15 to 25 cycles at 94° C., 15 sec; 54° C., 20 sec; 72° C., 1 to 1.5 min, followed by a final extension of 5 min at 72° C.

Oligonucleotide design was as indicated in Table 4, and oligonucleotides were supplied by Sigma Genosys Ltd (Haverhill, UK) or by GeneLink Inc. (Hawthorn, N.Y., USA). Library primers were designed to alter the appropriate sequence of the Pin1 WW domain as described above. The tac-PinLib-RepA-CIS-ori PCR construct was prepared as follows: the RepA-CIS-ori region was amplified by PCR from the R1 plasmid [GenBank accession no. V00351] using primers 1StepRepFor (SEQ ID NO: 4) and M13rev (SEQ ID NO: 5). A second PCR was then performed on RepA-CIS-ori with a long primer encoding the Pin1 domain and GSGSS linker (SEQ ID NO: 13) and primer DigLigRev (SEQ ID NO: 6). In this second PCR, 1× ThermPol buffer was replaced with 1.5× Standard Buffer and the elongation temperature was set at 68° C. Finally, the tac promoter was appended upstream of the nucleic acid framework library in a third PCR using primers 131 mer (SEQ ID NO: 7) and DigLigRev as before. PCR products were purified using a Wizard PCR Cleanup kit following manufacturers recommended procedures (Promega UK Ltd, Southampton, UK).

TABLE 4
WW domain framework peptide sequences
and oligonucleotides used in
construction of framework library.

Pin1 WW domain amino acid sequence
(SEQ ID NO: 1):
MADEEKLPPGWEKRMSRSSGRVYYFNHITNASQWERPSG
Mutated Pin1 WW domain amino acid sequence
(SEQ ID NO: 12):
MADEEKLPPGWEKRWSRS-GRVYYFNHITNASQWERPSG
Pin1 WW domain framework peptide library
sequence (SEQ ID NO: 2):
MADEEKLPPGWX₁KX₂WSX₃X₄GRVX₅YX₆NX₇ITX₈AX₉QWERPSG
Pin1 WW domain framework peptide and linker
(SEQ ID NO: 14):
MADEEKLPPGWX₁KX₂WSX₃X₄GRVX₅YX₆NX₇ITX₈AX₉QWERPSGGS
GSS
1StepRepFor (SEQ ID NO: 4):
5′- GGCAGCGGTTCTAGTCTAGCGGCCCCAACTGATCTTCACCAAACG
TATTACC -3′
M13rev (SEQ ID NO: 5):
5′- CAGGAAACAGCTATGAC-3′
Pin1 WW domain framework library (SEQ ID NO: 3):
5′- ATGGCCGATGAAGAGAAACTGCCGCCAGGCTGGNNBAAANNBTGG
AGTWMVVMGGACGCGTCNNBTACNNBAATNNBATCACTNNBGCGVVMCA
GTGGGAACGACCATCGGGC -3′
Pin1 WW domain framework library and linker
(SEQ ID NO: 13):
5′- GGAAACAGGATCTACCATGGCCGATGAAGAGAAACTGCCGCCAGG
CTGGNNBAAANNBTGGAGTVVMVVMGGACGCGTCNNBTACNNBAATNNB
ATCACTNNBGCGVVMCAGTGGGAACGACCATCGGGCGGCAGCGGTTCTA
GTCTAGC -3′
DigLigRey (SEQ ID NO: 6):
5′- CATGATTACGCCAAGCTCAGAA -3′
131 mer (SEQ ID NO: 7):
5′- CGGCGGTTAGAACGCGGCTACAATTAATACATAACCCCATCCCCC
TGTTGACAATTAATCATCGGCTCGTATAATGTGTGGAATTGTGAGCGGA
TAACAATTTCACACAGGAAACAGGATCTACCATGGCC -3′
Key:
N = G, A, T, C
B = G, T, C
V = A, C, G
M = A, C

To verify the library sequence, a fraction of the library was cloned in a TOPO-TA vector (Invitrogen), transformed into One Shot TOP10 bacterial cells (Invitrogen), and single clones were analysed by sequencing. Sequences showed the expected randomisation at the mutated positions.

### Example 2

### A. Selection Against an Extracellular Target—VEGFR2

Generally, in vitro transcription and translations were carried out as described by Odegrip et al. (2004, Proc. Natl. Acad. Sci. USA, 101, 2806-2810). 20 μg of each library DNA template (encoding approximately 10¹³ library members) was added to 200 μl in vitro transcription and translation (ITT) mixture of an S30 lysate system for linear templates (Promega UK Ltd, Southampton, UK). Incubation was performed for up to 60 min at 30° C. in order to generate protein-nucleic acid complexes. After expression, the samples were diluted 5-fold in Selection Buffer containing 2% BSA (Sigma Aldrich Company Ltd, Gillingham, UK), 0.1 mg/ml herring sperm DNA (Promega UK Ltd, Southampton, UK), in PBS (Dulbecco A, Oxoid, Basingstoke, UK).

2 μg of VEGFR2-Fc fusion (VEGFR2; R&D Systems, Abingdon, Oxfordshire, UK; SEQ ID NO: 8; Table 5) comprising the VEGFR2 target ligand was immobilised on 20 μl TALON® magnetic beads (Life Technologies, Paisley, UK) in PBS. Coated beads were washed three times with 1 ml PBS-T (PBS, 0.1% Tween-20) and twice with PBS. The diluted ITT reactions were added to the coated TALON® magnetic beads and incubated for 1 hour at room temperature whilst mixing on a rotary mixer. The beads were then removed from the Selection Buffer and washed 4 times with PBS-T, and once with PBS (5 min/wash). Bound DNA was eluted from the beads by incubation at 65° C. in 100 μl of 1× ThermoPol buffer (NEB Ltd., Hitchin, UK) and heated for 10 min. Half of the eluted material was added to a recovery PCR reaction where the N-terminal library region was amplified using primers R1RecFor (SEQ ID NO: 9) and NotRecRev (SEQ ID NO: 10) shown in Table 6. All recovered PCR product was reattached to the RepA-CIS-ori by ligation, as described in Odegrip et al. (2004), and further amplified with primers R1RecFor and DigLigRev (Table 4), thereby producing input DNA for the next round of selection.

For subsequent rounds of expression, screening and selection, the resulting DNA from a preceding round was added to a fresh ITT mixture and the selection process was repeated. In this example, the process was repeated three times (i.e. a total of 4 rounds of expression, screening and selection), to enrich the pool of binding clones. To increase the selection pressure in the second and subsequent rounds of selection and favour the most desirable peptides, selection conditions were modified as shown in Table 6. In this example, after the first round of selection the length of the washes after incubation of the coated beads with ITT were increased for each round. In the third round the target VEGFR2 ligand concentration was decreased to favour high affinity binders.

TABLE 5
Oligonucleotide and peptide sequences used in selection procedure.

VEGFR2-Fc fusion (SEQ ID NO: 8):
ASVGLPSVSLDLPRLSIQKDILTIKANTTLQITCRGQRDLDWLWPNNQSGSEQRVEVTE
CSDGLFCKTLTIPKVIGNDTGAYKCFYRETDLASVIYVYVQDYRSPFIASVSDQHGVVYIT
ENKNKTVVIPCLGSISNLNVSLCARYPEKRFVPDGNRISWDSKKGFTIPSYMISYAGMVF
CEAKINDESYQSIMYIVVVVGYRIYDVVLSPSHGIELSVGEKLVLNCTARTELNVGIDFNW
EYPSSKHQHKKLVNRDLKTQSGSEMKKFLSTLTIDGVTRSDQGLYTCAASSGLMTKKN
STFVRVHEKPFVAFGSGMESLVEATVGERVRIPAKYLGYPPPEIKWYKNGIPLESNHTIK
AGHVLTIMEVSERDTGNYTVILTNPISKEKQSHVVSLVVYVPPQIGEKSLISPVDSYQYGT
TQTLTCTVYAIPPPHHIHWYWQLEEECANEPSQAVSVTNPYPCEEWRSVEDFQGGNKI
EVNKNQFALIEGKNKTVSTLVIQAANVSALYKCEAVNKVGRGERVISFHVTRGPEITLQP
DMQPTEQESVSLWCTADRSTFENLTWYKLGPQPLPIHVGELPTPVCKNLDTLWKLNAT
MFSNSTNDILIMELKNASLQDQGDYVCLAQDRKTKKRHCVVRQLTVLERVAPTITGNLE
NQTTSIGESIEVSCTASGNPPPQIMWFKDNIEGRMDPKSCDKTHTCPPCPAPELLGGPS
VFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKS
RWQQGNVFSCSVMHEALHNHYTQKSLSLSPGK
R1recfor (SEQ ID NO: 9):
5′- GAACGCGGCTACAATTAATACATAACC -3′
TAC6 (SEQ ID NO: 11):
5′- CCCCATCCCCCTGTTGACAATTAATC -3′
NOT1RECREV (SEQ ID NO: 10):
5′- TGGTGAAGATCAGTTGCGGCCGCTAG -3′

To help maintain the accuracy of PCR product sequences nested forward primers may be used. In this example, primer R1RecFor was replaced by Tac6 (SEQ ID NO: 11) in the third and fourth rounds of the selection procedure (Table 5).

TABLE 6
Conditions used in selection against VEGFR2

| | Beads | Target | Washes |
|---|---|---|---|
| Round | (TALON) | (VEGFR2) | (4xT-PBS + 1xPBS) |
| 1 | 20 μl | 2 μg | 5 min each |
| 2 | 20 μl | 2 μg | 7 min each |
| 3 | 10 μl | 1 μg | 8 min each |
| 4 | 10 μl | 1 μg | 5 min each |

### B. Binding Analysis by ELISA

The output from last round of selection was digested with NotI and NcoI and ligated into a similarly digested M13 gplll phagemid vector. The resulting DNA was transformed into E. coli TG-1 cells, and plated on 2% glucose, 2×TY, 100 μg/ml ampicillin plates. Individual colonies were grown for the production of phage particles, using a helper phage (M13K07; NEB Ltd, Hitchin, UK). The methods were performed generally as described in Odegrip et al. 2004, Proc. Natl. Acad. Sci. USA, 101 2806-2810

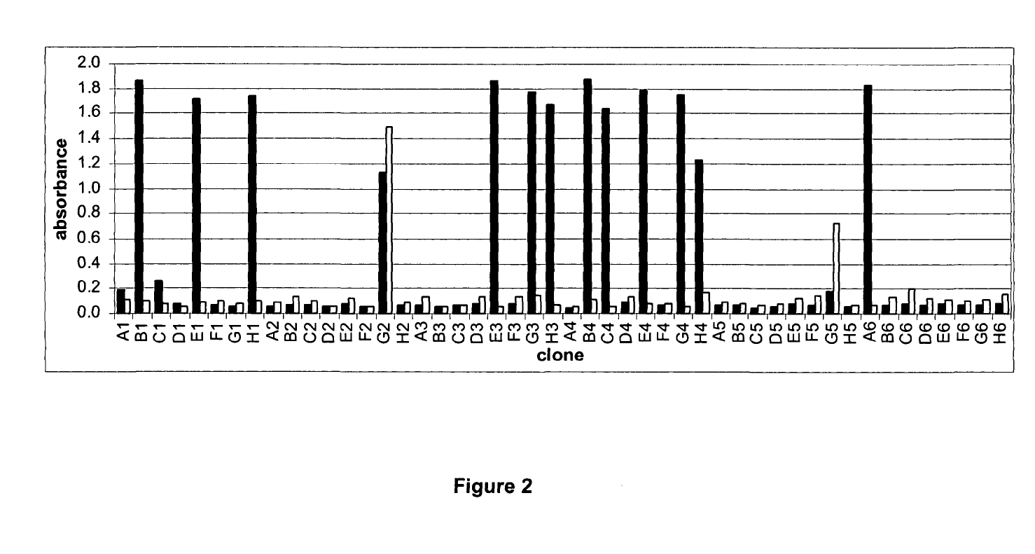

ELISAs were then performed to screen for modified WW domain peptides that bind to the target ligand. NUNC Maxisorp plates were coated with 50 ng per well of VEGFR2 in PBS overnight at 4° C. After blocking the plates with PBS, 2% BSA, 0.1% Tween-20, phage displaying the selected clones were added, diluted in blocking buffer, and incubated for 1 hour. WW domain binders were detected using horseradish peroxidase-conjugated anti-M13 antibody (GE) and SureBlue TMB peroxidase substrate (Insight Biotechnology, Middlesex, UK); and the ELISA signal was read at 450 nm. An ELISA screen of some of the clones from the output of the selection against VEGFR2 showed that the signal obtained on VEGFR2 was far greater than on a microtitre plate blocked with BSA (FIG. 2).

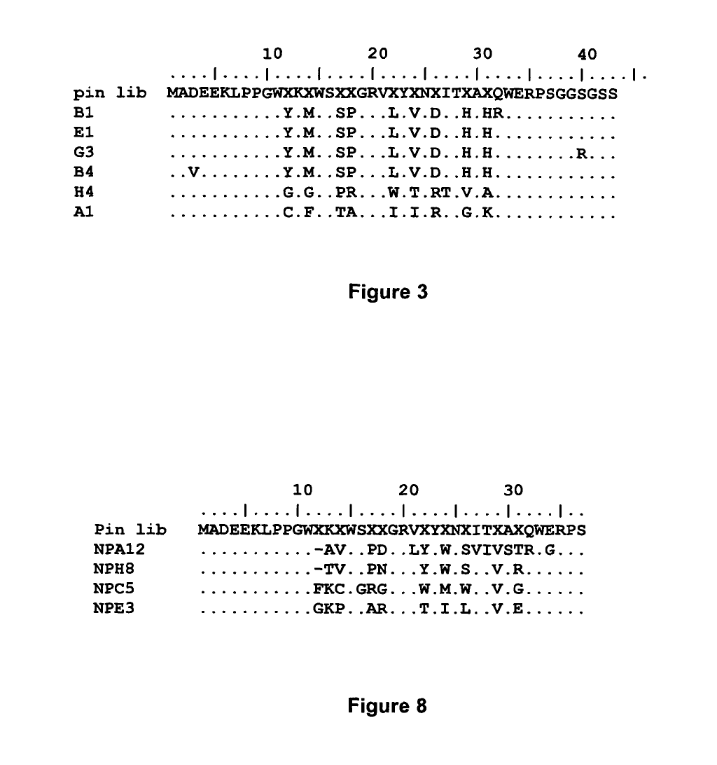

A selection of clones showing a high signal on VEGFR2 was analysed by sequencing the corresponding nucleic acids to obtain modified WW domain peptide sequences (Table 7 and FIG. 3). Interestingly, clones B1 and H1 were found to have identical sequences (SEQ ID NO: 15). Clones E1, E3, H3, C4, E4 G4 and H6 were also found to be identical (SEQ ID NO: 16). Clones G3 (SEQ ID NO: 17), B4 (SEQ ID NO: 18), H4 (SEQ ID NO: 19) and A1 (SEQ ID NO: 20) were unique.

TABLE 7
Modified Pin1 WW domain peptides that bind VEGFR2.

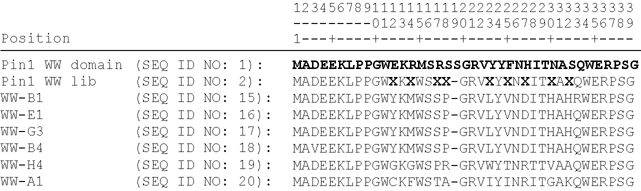

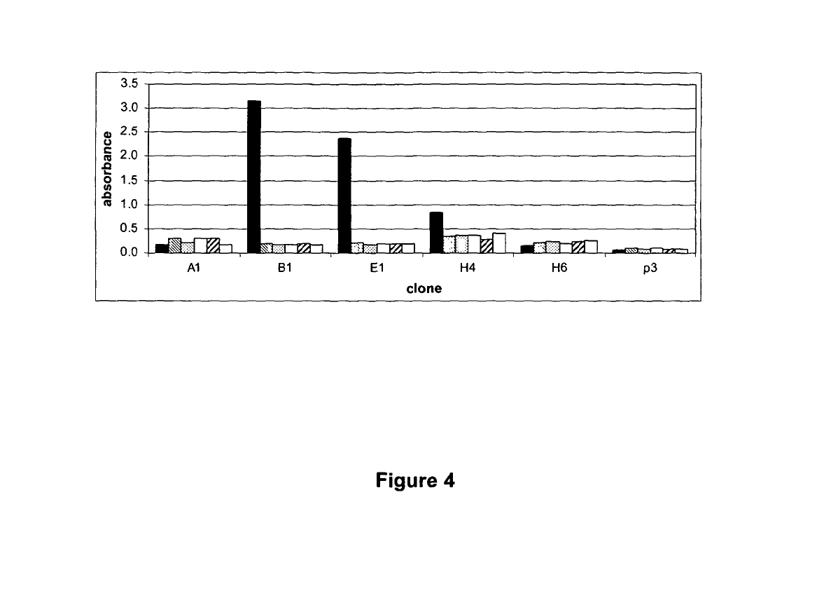

The specificity of clones selected for binding to VEGFR2 was assayed in an ELISA against a selection of non-target peptides/proteins, as described above. 50 ng of each of the different target and non-target proteins was coated per well of and ELISA plate and each of the modified WW domain peptides was added. The results demonstrate that the selected peptides are highly specific for VEGFR2 (FIG. 4).

The amino acid variants selected at each position indicated as ‘X’ in SEQ ID NO: 2 (i.e. X1 to X9, respectively from N to C-terminus) in the WW clone sequences shown in Table 7 represent defined sub-groups of preferred amino acids for each X position. Thus, for binding to VEGFR2, preferably in WW domain peptides and libraries of the invention derived from SEQ ID NO: 2: the X1 position may be randomly selected from amino acid residues Y, G and C; the X2 position may be randomly selected from amino acid residues M, G and F; the X3 position may be randomly selected from amino acid residues S, P and T; the X4 position may be randomly selected from amino acid residues P, R and A; the X5 position may be randomly selected from amino acid residues L, W and I; the X6 position may be randomly selected from amino acid residues V, T and I; the X7 position may be randomly selected from amino acid residues D and R; the X8 position may be randomly selected from amino acid residues H, V and G; and/or the X9 position may be randomly selected from amino acid residues H, A and K.

### C. Determination of Affinity for VEGFR2 Using Biolayer Interferometry (ForteBio).

The WW-B1 VEGFR2 WW domain was chemically synthesised. The peptide was synthesised using standard Fmoc/tBu protocols. The peptide chain was elongated on a tentagel resin (Intavis, Koeln, Germany, 0.24 mmol/g), using HBTU as a coupling reagent. All amino acids were introduced with standard protecting groups (Asp(OtBu), Glu(OtBu), Lys(Boc), Trp(Boc), Tyr(tBu), Ser(tBu), Thr(tBu), Arg(Pmc), Asn(Trt) and His(Trt): Merck, Nottingham, UK). After the final Fmoc deprotection, a solution of biotin (10 equivalents to peptidyl-resin), HBTU (10 equivalents to peptidyl-resin) and DIPEA (20 equivalents to peptidyl-resin) in 2 ml NMP/DMF (1:1) were added to the peptidyl-resin in 1 ml of dry DMF and mixed at RT for 1.5 hours. The solution was filtered off and the peptidyl-resin washed extensively with DMF and the DCM. 5 ml of cleavage mixture (TFA:TIS:H₂O as 95:2.5:2.5) was added to the peptidyl-resin and the reaction was left to proceed with shaking at room temperature for 2.5 hrs. After TBME precipitation and washes, the crude peptide was dissolved in H₂O and lyophilised. Purification of the peptide was carried out on a Perkin Elmer HPLC using a reverse-phase analytical C18 column and a gradient of 2% to 70% B into A over 60 minutes (A=100% H₂O, B=95% acetonitrile/5% H₂O; both contain 0.1% trifluoroacetic acid). The peptide was obtained as a white powder after a final lyophilisation step.

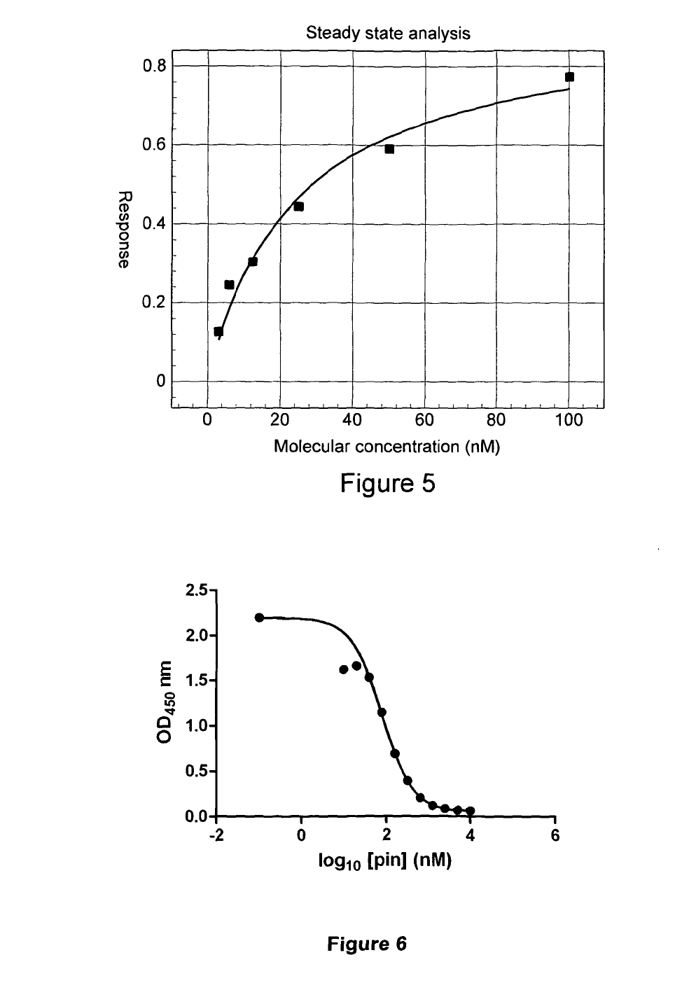

Real time binding assays between chemically synthesised clone WW-B1 and VEGFR2-Fc fusion were performed using biolayer interferometry with an Octet Red system (Fortebio, Menlo Park, Calif.). Biotinylated WW-B1 was immobilised to streptavidin biosensors (Fortebio) at a concentration of 50 ng/μl in PBS. Association curves were then detected by incubating WW-B1-coated sensors with different concentrations of VEGFR2 (1 to 100 nM in PBS), and dissociations were detected by incubating the sensors in PBS. The dissociation constant for WW-B1 binding to VEGFR2 was determined by steady state analysis to be 44 nM±1.1 nM (FIG. 5).

### D. Biological Activity Against an Extracellular Target—VEGFR2

A competition assay was carried out in which binding of VEGFR2 to VEGF-A (VEGF) was competed by the presence of varying amounts of the selected modified WW domain peptide.

50 ng human VEGF (R&D Systems, Abingdon, UK) 1 μg/ml in PBS was coated per well onto a Maxisorp plate at 37° C. for 140 minutes. The wells were then blocked with 100 μl PBS containing 2% BSA for 55 minutes at room temperature. 1.25 ng VEGFR2 was preincubated with WW-B1 fused to maltose binding protein (MBP) in 50 μl PBS for 45 minutes at room temperature. The activity of the WW-B1 peptide was tested at serial dilutions from 10 μM to 156.25 nM. 50 μl 4% BSA in PBS was added to the preincubated VEGFR2 with WW-B1-MBP and the whole mixture was then added to the VEGF coated ELISA plate and incubated at room temperature for 55 minutes. The plate was then washed with 3 washes in PBS-Tween and 2 washes in PBS. Next 50 μl/well penta His-HRP (QIAgen, diluted 1:500 in 2% BSA in PBS) was added and the mixture incubated at room temperature for 55 minutes, before washing as before. ELISA signals were detected using SureBlue TMB peroxidase substrate (Insight biotechnology, Middlesex, UK) at 450 nm.

As a control, the above process was repeated with wild-type Pin1 WW domain peptide replacing WW-B1. The results are illustrated in FIG. 6. These data demonstrate that the VEGFR2-binding WW domain peptide, WW-B1, strongly inhibits the binding of VEGFR2 to VEGF with an EC₅₀ value of 76 nM. In contrast, the wild-type Pin1 WW domain peptide has essentially no effect on the binding of VEGFR2 to VEGF and, hence, negligible binding affinity for VEGFR2.

Hence, these data show that WW domains are useful scaffolds for the selection of novel binding domains and for the creation of new protein-protein interactions. It has also been shown that WW domains can be engineered to bind target ligands that are not naturally recognised by WW domains, and furthermore, that such modified WW domains can bind these non-natural target ligands with high affinity. More specifically, it has been shown that a modified Group IV WW domain peptide can be engineered to bind non-phosphorylated and extracellular target ligands.

### Example 3

### A. Selection against a growth factor—β-NGF

Cloning, in vitro transcription and translations and screening and selection procedures were generally carried out as described in Examples 1 and 2 above.

Briefly, an initial DNA template library sample of approximately 10¹³ members was added to an ITT solution to generate protein-DNA complexes. After expression, the samples were diluted with blocking buffer (PBS, 2% BSA, 0.1 mg/ml herring sperm DNA, 1 mg/ml heparin). The blocking buffer and ITT (selection mixture) was incubated twice in Immunotubes (Nunc) for 30 minutes and in each instance the supernatant was retained. The selection mixture was then incubated twice with streptavidin beads (Dynabeads M-280, Life Technologies, Paisley, UK) for 30 minutes and the beads removed using a magnet to remove any streptavidin binders. Again, the supernatant was retained.

Human β-NGF (SEQ ID NO: 21; Table 8; R&D Sytems, Abingdon, UK) was biotinylated via carboxyl group substitution (Rosenberg et al., 1986, J. Neurochem. 46, 641-8) and added to the above supernatant to give a final concentration of 1 μg/ml. The mixture was incubated at room temperature for 1 hour. To this mixture, 10 μl of streptavidin-coated beads were added and incubated, whilst mixing, for 10-15 minutes at room temperature. The beads were washed with four washes of 1 ml PBS-T and two washes of PBS, and the tubes were changed twice during the procedure. The WW domain peptide-bound beads were then resuspended in 60 μl HF buffer (New England Biolabs, Hitchin, UK), and heated for 10 minutes at 75° C. 5 μl of the eluted material and beads was used as a template for PCR in a 50 μl reaction. Half of the eluted material was added to a recovery PCR reaction where the N-terminal library region was amplified by using primers R1RecFor and NotRecRev for 35 cycles. All recovered PCR product was reattached to the RepA-CIS-ori by ligation and further amplified with primers Tac6 (SEQ ID NO: 11) and DigLigRev (SEQ ID NO: 6) as previously described.

TABLE 8
β-NGF peptide sequence and PCR primers for
library selections.

Human β-NGF peptide sequence (SEQ ID NO: 21):
SSHPIFHRGEFSVCDSVSVWVGDKTTATDIKGKEVMVLGEVNINNSVFK
QYFFETKCRDPNPVDSGCRGIDSKHWNSYCTTTHTFVKALTMDGKQAAW
RFIRIDTACVCVLSRKAVRR
NotRecRevWW1 (SEQ ID NO: 22):
5′- AGATCAGTTGCGGCCGCAGAGCTTGCTACGGTGCTGCCCGATGGT
CGTTCCCAC -3′
Tac6-9 (SEQ ID NO: 23):
5′- CGGCTCGTATAATGTGTGGAATTGTGAGC -3′
NotRecRevWW2 (SEQ ID NO: 24):
5′- AGATCAGTTGCGGCCGCAGAGCTTGC -3′
110-mer (SEQ ID NO: 25):
5′- CGGCGGTTAGAACGCGGCTACAATTAATACATAACCCCATCCCCC
TGTTGACAATTAATCATGGCTCGTATAATGTGTGGAATTGTGAGCGGAT
AACAATTTCACACAGG-3′

DNA PCR products were then added to an ITT solution and the selection process was repeated three times to enrich the pool of binding clones using the quantities described in Table 9. The second round was recovered with Tac6 (SEQ ID NO: 11) and NotRecRevWW1 (SEQ ID NO: 22) primers and reamplified following RepA-CIS-ori ligation with Tac6 (SEQ ID NO: 11) and DigLigRev (SEQ ID NO: 6) primers. Round 3 DNA was recovered with Tac6-9 (SEQ ID NO: 23) and NotRecRevWW2 (SEQ ID NO: 24) primers and reamplified following RepA-CIS-ori ligation with 110-mer (SEQ ID NO: 25) and DigLigRev (SEQ ID NO: 6). Round 4 was recovered with R1RecFor (SEQ ID NO: 9) and NotRecRevWW2 (SEQ ID NO: 22) primers. Primer sequences are shown in Table 8.

TABLE 9
Conditions used in selection against NGF

| | Beads | Target | Washes |
|---|---|---|---|
| Round | (M-280) | (β-NGF) | (T-PBS + PBS) |
| 1 | 10 μl | 1.5 μg | 4 + 2 |
| 2 | 10 μl | 0.5 μg | 5 + 1 |
| 3 | 10 μl | 0.25 μg | 6 + 1 |
| 4 | 10 μl | 0.25 μg | 7 + 1 |

### B. Binding Analysis by ELISA

The output DNA from the last round of selection was cloned into M13 gpIII phagemid vector, transformed into E. coli TG-1 cells, plated on 2% glucose, 2×TY, 100 μg/ml ampicillin plates for the production of phage particles, using a helper phage (M13K07; NEB Ltd, Hitchin, UK) and colonies were screened as previously described.

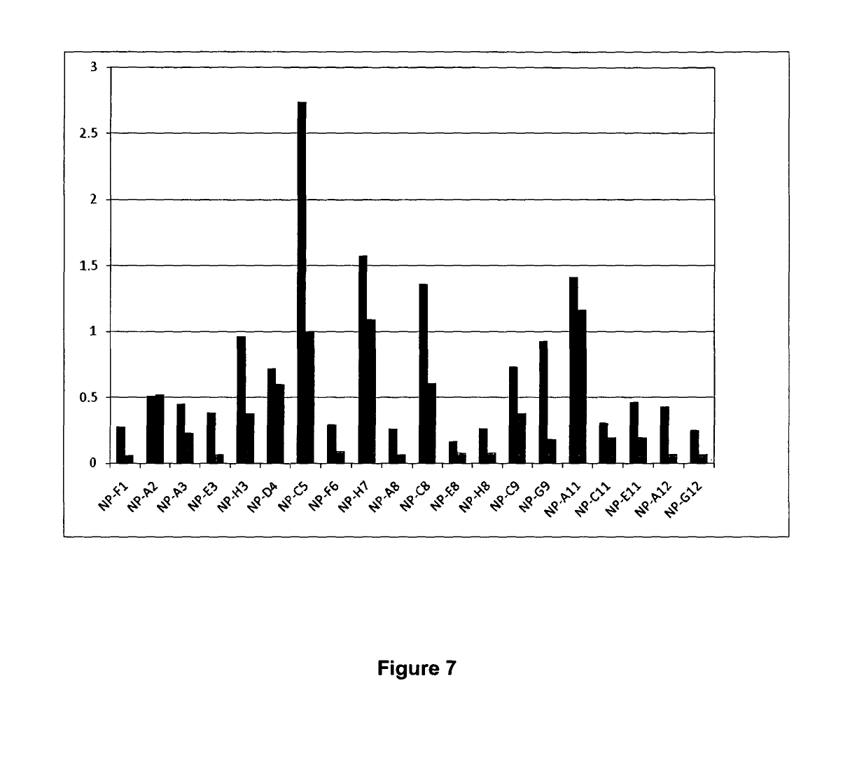

The binding specificities of the selected modified WW domain peptides were assayed by comparison to binding affinity for HSA, in a similar manner to that for VEGFR2 above and the results are shown in FIG. 7. Selected clones showing a high binding signal against β-NGF were then analysed by sequencing (Table 10, FIG. 8). Interestingly, the modified WW domain peptide clones NP-A12 (SEQ ID NO: 26) and NP-H8 (SEQ ID NO: 27) each had an amino acid deletion at amino acid position 12 of the wild-type Pin1 WW domain sequence, thereby shortening the first strand of the WW domain by one amino acid.

TABLE 10
Modified Pin1 WW domain peptides that bind β-NGF.

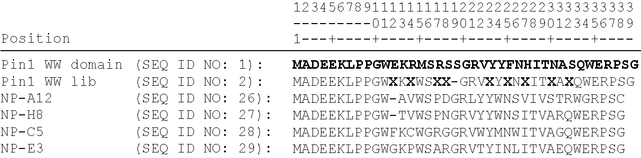

The amino acid variants selected at each position indicated as ‘X’ in SEQ ID NO: 2 (i.e. X1 to X9, respectively from N to C-terminus) in the WW clone sequences shown in Table 10 represent defined sub-groups of preferred amino acids for each X position. Thus, for binding to β-NGF, preferably in WW domain peptides and libraries of the invention derived from SEQ ID NO: 2: the X1 position may be randomly selected from an amino acid deletion or amino acid residues F and G; the X2 position may be randomly selected from amino acid residues V, C and P; the X3 position may be randomly selected from amino acid residues P, R and A; the X4 position may be randomly selected from amino acid residues D, N, G and R; the X5 position may be Y; the X6 position may be N; the X7 position may be randomly selected from amino acid residues V and I; the X8 position may be randomly selected from amino acid residues S and A; and/or the X9 position may be randomly selected from amino acid residues R and Q.

### Example 4

### A. Cyclisation of Modified WW Domain Peptides

For use as therapeutic agents, as well as in other situations, it may be advantageous to cyclise the modified WW domain peptides, for example, to improve stability, resistance to proteases etc., while maintaining the desired biological activity of the original linear peptide.

Two cyclic modified WW domain peptides: Biotin-DEEKLPPGWYKMWSSPGRVLYVNDITHAHRWERPEG-NH₂ (SEQ ID NO: 30); and Biotin-DEEKLPPGWYKMWSSPGRVLYVNDIKHAHRWERPEG-NH₂ (SEQ ID NO: 34)—where the underlined portion indicates the cyclised region), were generated based on the WW-B1 peptide identified in Example 2. As indicated, the cyclic peptides are amidated at their C-terminus and biotinylated at their N-terminus in order to help capture the peptide for affinity analysis.

The sequence of WW-B1 (SEQ ID NO: 15) was mutated to substitute the serine at position 38 to glutamate (Ser38Glu), to enable a covalent bond to be formed between the side-chains of lysine at position 6 and glutamate at position 38 for peptide SEQ ID NO: 30. For peptide SEQ ID NO: 34, on top of the aforementioned Ser38Glu substitution, the threonine at position 29 was substituted for a lysine, this time to enable a covalent bond to be formed between the side-chains of glutamate at position 4 and lysine at position 29. Furthermore, since the peptides were to be chemically synthesised, the N-terminal dipeptide Met-Ala was not required for cloning or expression, and so the modified cyclic Pin1 WW domain peptide sequence begins at position 3 of SEQ ID NO: 15. The synthesis process involved a number of steps, as indicated below.

### Chain Elongation:

The peptides were synthesised using standard Fmoc/tBu protocols. Peptide chains were elongated on a tentagel resin (Intavis, Koeln, Germany, 0.24 mmol/g), using HBTU as a coupling reagent. Amino acids were introduced with the following side chain protecting groups: Asp(OtBu), Glu(OtBu), Lys(Boc), Trp(Boc), Tyr(tBu), Ser(tBu), Thr(tBu), Arg(Pmc), Asn(Trt), and His(Trt) apart from the following residues: for peptide SEQ ID NO: 30, lysine residue at position 6 according to the numbering of SEQ ID NO: 1 (position 4 in SEQ ID NO: 30) was introduced using Fmoc-Lys(Mtt)-OH, and the glutamic acid residue at position 38 according to the numbering of SEQ ID NO: 1 (position 35 of SEQ ID NO: 30) was introduced as Fmoc-Glu(OAII)-OH (Merck, Nottingham, UK). For peptide SEQ ID NO: 34, the lysine residue at position 29 according to the numbering of SEQ ID NO: 1 (position 26 of SEQ ID NO: 34) and the glutamic acid residue at position 4 according to the numbering of SEQ ID NO: 1 (position 2 of SEQ ID NO: 34) were introduced using the allyl-based protecting groups, Fmoc-Lys(Alloc)-OH and Fmoc-Glu(OAII)-OH (Merck, Nottingham, UK).

### Biotinylation:

After the final Fmoc deprotection, a solution of biotin (10 equivalents to peptidyl-resin), HBTU (10 equivalents to peptidyl-resin) and DIPEA (20 equivalents to peptidyl-resin) in NMP/DMF (1:1) was added to the peptidyl-resins and mixed at RT for 1.5 hours. The solution was filtered off and the peptidyl-resins washed extensively with DMF and DCM.

### OAII Deprotection:

Pd(PPh₃)₄ (5 equivalents to peptidyl-resin) was dissolved in 10 ml of CHCl₃/AcOH/NMM (9.25:0.5:0.25). This solution was added to the peptidyl-resin and left to stir at room temperature for 2.5 hours for peptide SEQ ID NO: 30 and at 45° C. for 1.5 hrs for peptide SEQ ID NO: 34. Washes with DIPEA in DMF (0.5% v/v) were followed by washes with NaCS₂N(C₂H₅)₂.3H₂O (sodium diethyldithiocarbamate) in DMF (0.5% w/v) and finally DMF and DCM washes.

### Mtt Deprotection: For Peptide SEQ ID NO: 30 Only

A solution of 1% TFA, 5% TIS in DCM was added to the peptidyl-resin, shaken for 2 minutes, and then filtered off. This step was repeated 15-20 times, after which DCM washes were completed.

### Cyclisation:

The peptidyl-resin were swelled in dry DMF for 10-15 minutes. HATU (5 equivalents to peptidyl-resin) was dissolved into dry DMF and this solution was added to the peptidyl-resins. DIPEA was added to this (10 equivalents to peptidyl-resin) and the reactions were left at room temperature for 15 hours. The solution was then filtered off and the resins washed with DMF and DCM.

Cleavage from Resin:

TFA cleavage mixture (TFA:TIS:H₂O as 95:2.5:2.5) was added to the peptidyl-resins and the reactions were left to proceed with shaking at room temperature for 2.5 hrs. After TBME precipitation and washes, the crude peptide was dissolved in H₂O and lyophilized.

### Purification:

Purification of the peptides was carried out on Agilent 1100 series HPLC using a reverse-phase analytical C18 column and a gradient of B into A (A=100% H₂O, B=95% acetonitrile/5% H₂O; both contain 0.1% trifluoroacetic acid). Peptides were obtained as white powders after a final lyophilisation step.

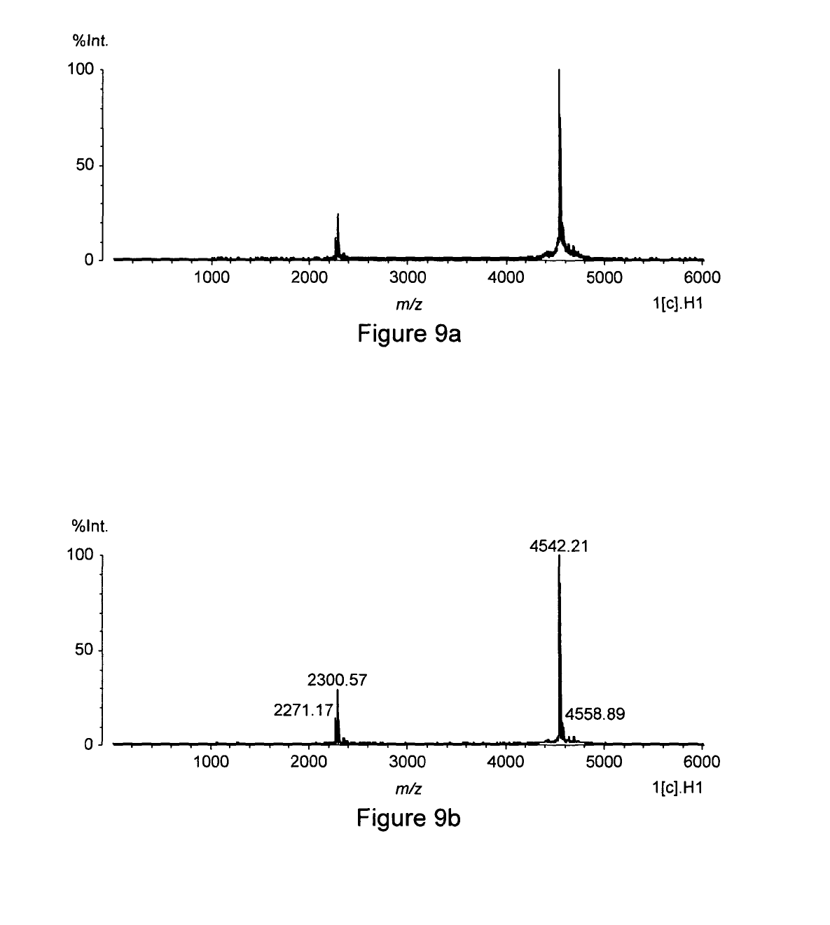

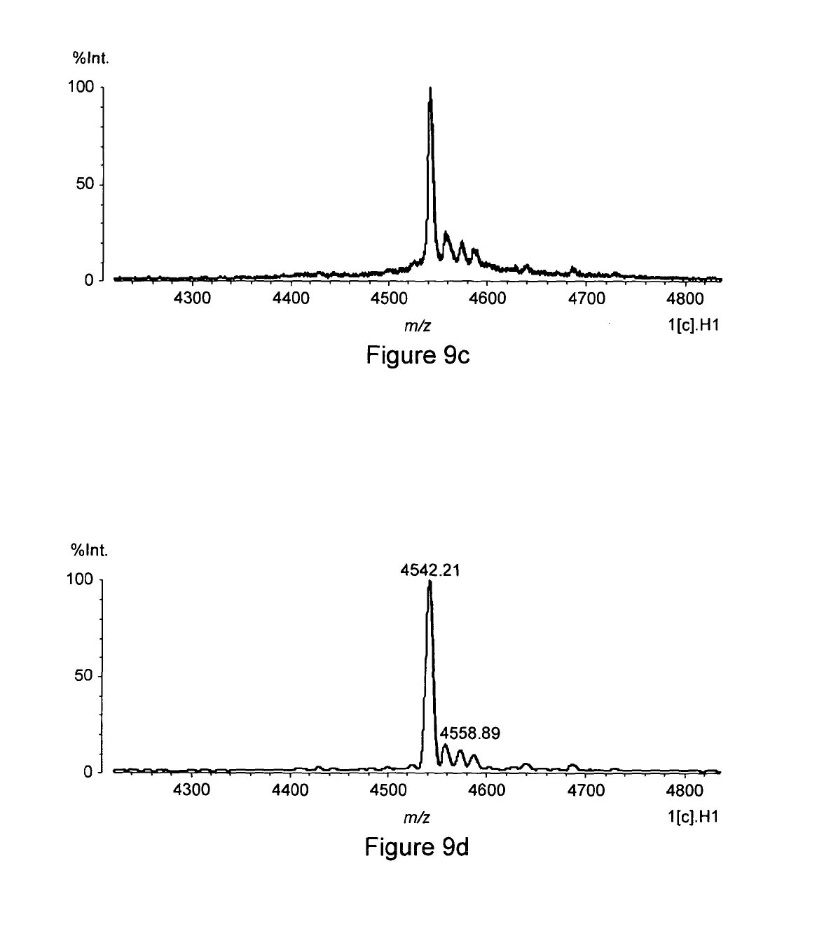

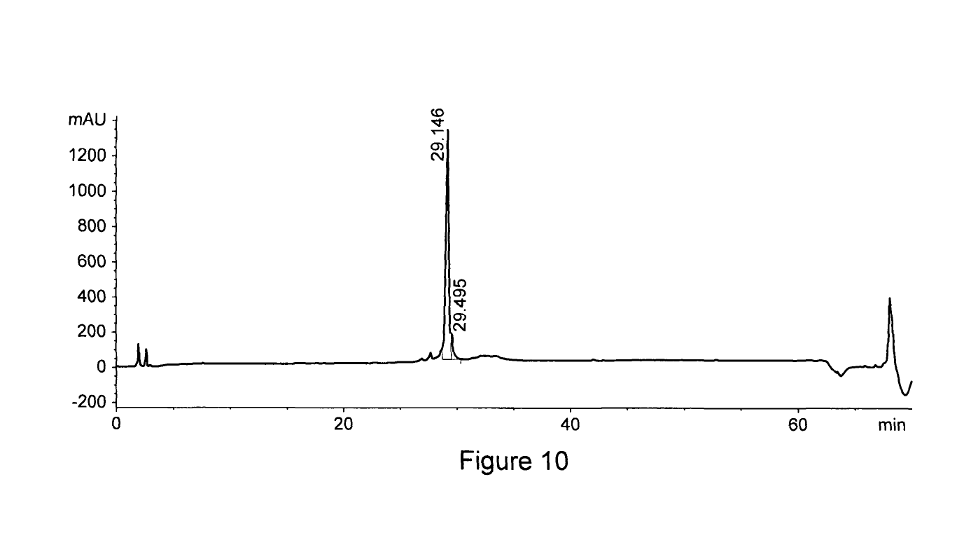

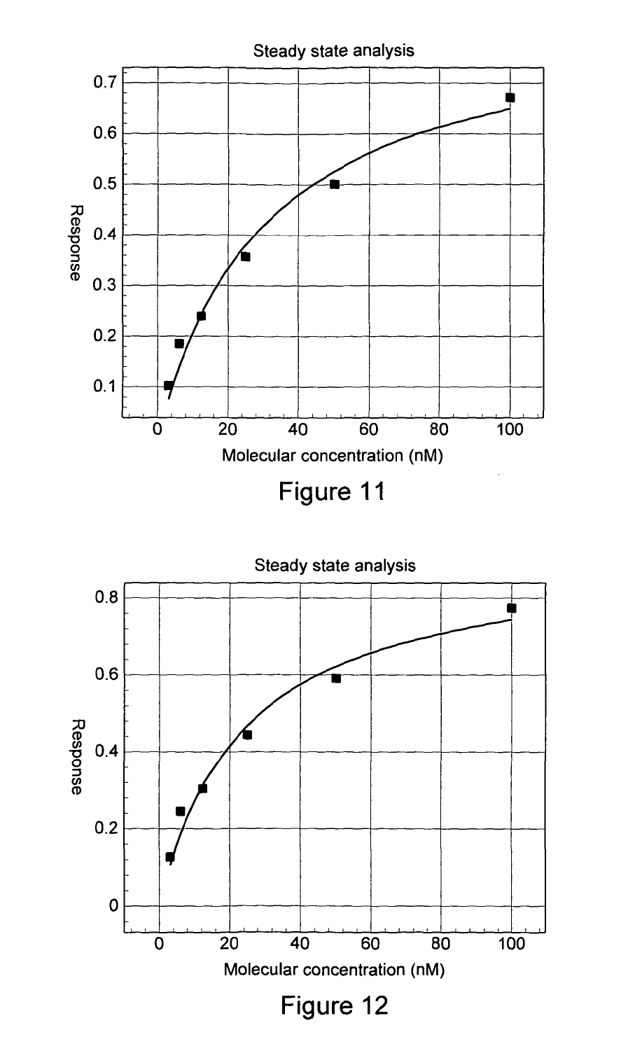

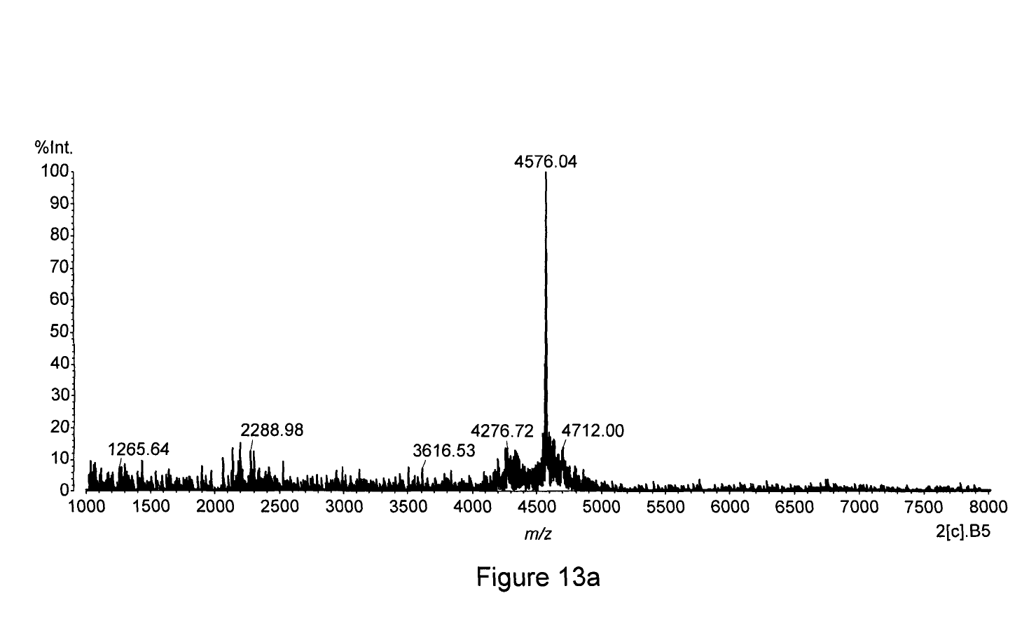

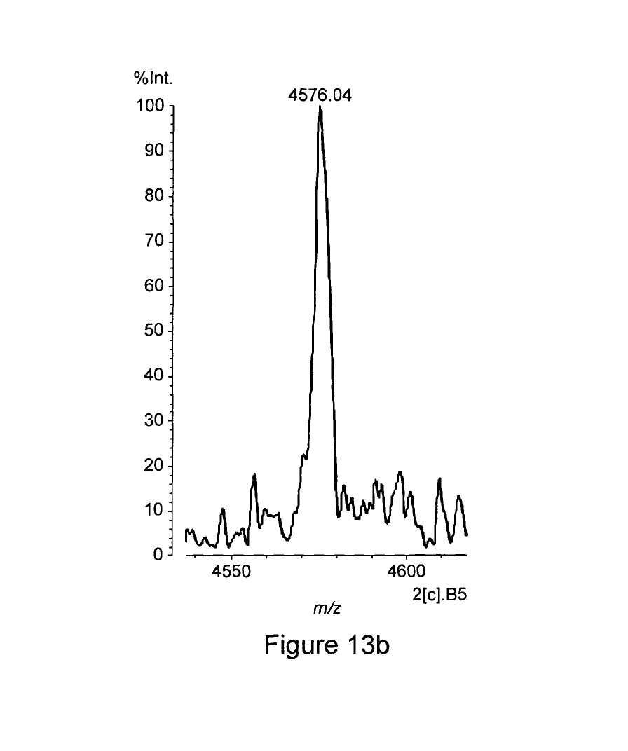

The identity of the peptides was confirmed using MALDI-TOF mass spectrometry (Axima, Shimadzu Biotech, Milton Keynes, UK), and the results are shown in FIG. 9 (peptide of SEQ ID NO: 30), and in FIG. 13 (peptide of SEQ ID NO: 34). For peptide SEQ ID NO: 30, the expected mass is 4540, which corresponds to the major peak found at 4542 (FIG. 9). For peptide SEQ ID NO: 34, the expected mass is 4574, which corresponds to the major peak found at 4576 (FIG. 13). Purity was assessed by using an analytical RP C18 column and found to be >90%, as shown in FIG. 10 for peptide SEQ ID NO: 30.

Cyclic peptide SEQ ID NO: 30 was tested for its binding affinity to VEGFR2 as set out in Example 2 above, and found to have comparable activity to the non-cyclised WW-B1 domain peptide of SEQ ID NO: 15.

### B. Biolayer Interferometry on Peptides

Real-time binding assays between the peptides and VEGFR2-Fc fusion were performed using biolayer interferometry with an Octet Red system (Fortebio, Menlo Park, Calif.). WW peptides were immobilised on streptavidin biosensors (Fortebio) at a concentration of 2 μg/ml in kinetic buffer. Association curves were then detected by incubating the immobilised peptide with streptavidin-HRP (1 to 100 nM in kinetic buffer), and dissociations were detected by incubating the sensors in kinetic buffer. All of the peptides from the affinity maturation, bind to VEGFR2 with a similar affinity as WW-B1.

The dissociation constant for cyclised WW-B1 binding to VEGFR2 was determined by steady state analysis to be 31 nM±4.8 nM (FIG. 12). The non-cyclised variant containing glutamic acid at position 38 according to the numbering of SEQ ID NO: 1 was also studied and had an affinity of 24 nM±4.0 nM for VEGFR2 (FIG. 13).

### Example 5

### A. Plasma Stability Assay

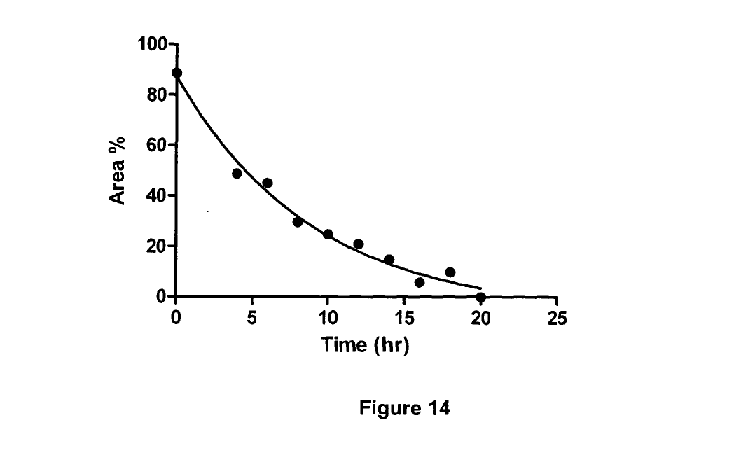

Peptides were resuspended in Molecular Biology Grade water at a concentration of 3 mM. The WW-B1 peptide was added to 300 μl mouse plasma (Harlan) or PBS (control) to a final concentration of 250 μM (2 samples). The samples were incubated at 37° C. 40 μl aliquots were removed from sample A at 0, 12, 14, 16, 18, 20 and 22 hr and from sample B at 0, 2, 4, 6, 8 and 10 hr. To purify each aliquot, double the volume of 100% ethanol (80 μl) was added and incubated at −20° C. for a few hours or overnight. The samples were centrifuged and 80 μl supernatant recovered. 40 μl water with 0.1% TFA was added to each sample and 100 μl was injected onto a SunFire C18 HPLC column. Buffer A (0.1% TFA in Water) was run through the column followed by 10-50% Buffer B (0.1% TFA, 5% water in 95% acetonitrile) over 20 min. The fractions from the column were collected and analysed using an AXIMA mass spectrometer. The half-life was calculated to be 6.2 hours using the one phase exponential decay equation in Prism (GraphPad Software Inc.; see FIG. 14).

### Example 6

### A. Incorporation of Non-Natural Amino Acids into WW Peptides

This example describes how a non-natural amino acid can be incorporated into the WW domain peptide sequences using CloverDirect™ Biotin-XX-AF (amber; Cosmo Bio Co. Ltd., Tokyo, Japan). This reagent has a unnatural aminoacyl-tRNA containing CUA anticodon and biotin.

A 50 μl in vitro transcription/translation reaction was set up using 10 μl DNA template encoding a tac promoter, WW-B1 (containing a TAG codon after the ATG methionine codon), repA, CIS and on (200 ng/μl), 20 μl 2.5× buffer, 15 μl S30 lysate and 1 μl of complete amino acid mixture containing 1 mM of each amino acid, 1 μl CloverDirect™ Biotin-XX-AF (amber) and 3 μl nuclease free water. This mixture was incubated at 30° C. for 60 minutes followed by incubation on ice. The CloverDirect™ Biotin-XX-AF (amber) tRNA was removed by further incubation with 5 units RNase ONE™ (Promega UK, Southampton, UK) for 5 minutes at 37° C. 5 μl reaction product was analysed by ELISA using immobilised VEGFR2 (as described in Example 1B) and Streptavidin HRP conjugate as a detection reagent for biotin (Thermo Scientific, Cramlington, UK) diluted 1:2000 dilution (0.625 μg/ml) in PBS containing 0.1% Tween. This was incubated for 60 minutes at room temperature followed by washing and developing conditions as above.

### Example 7

### A. CD Analysis

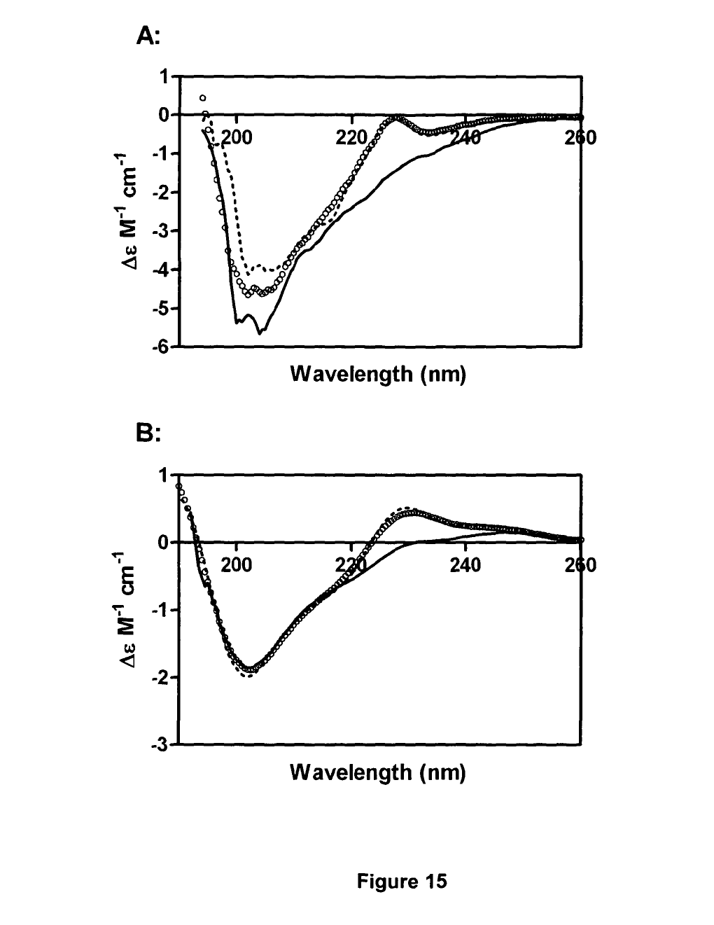

Peptides were resuspended in Molecular Biology Grade water or 10 mM sodium phosphate, pH 7, at a concentration of 200 μM to 1.4 mM. Far-UV scans were measured on an Aviv Model 410 spectrometer measuring at 0.5 nm intervals with a 1 sec averaging time, between 190 and 260 nm using 0.2 mg/ml peptide and a 1 nm bandwidth. Three scans were taken in total at each temperature and the results averaged. The CD scans were plotted using Aviv model 4.10 software, and the data were converted to molar ellipticity (M⁻¹ cm⁻¹). WW-B1 and cyclic WW-B1 both demonstrated CD spectra characteristic of WW domains having a peak at 227-231 nm and a trough at 202-204 nm at 25° C. The peak is attributable to the aromatic content of the peptide in a folded conformation and notably disappears at 95° C., which demonstrates unfolding at this temperature. However, the peptide is able to reform its structure after heating to 95° C. and cooling to 25° C. This far-UV spectrum is characteristic of other small structurally characterised β-sheet structures (Koepf et al. 1999, Protein Science, 8, 841-853; see FIG. 15).

### SUMMARY AND CONCLUSIONS

In summary, these tests demonstrate that the WW domain is a useful framework, template or scaffold for the selection of novel binding modules based on the WW domain. The WW domain framework of the invention is thus a useful framework for selecting binding modules against any possible target ligand, including nucleic acids (DNA or RNA), proteins and peptides (linear or conformation), and small molecules, such as organic or inorganic molecules. In short, the WW domain framework of the invention is thought to have applications where engineered antibodies have previously been used. In particular, the WW domain frameworks of the invention will have applications in selecting novel binding modules for protein-protein interactions.

In these studies it has been shown for the first time, that an engineered (i.e. modified) WW domain can bind to a target ligand that is not the natural target of, and is not bound by, the corresponding wild-type WW domain. Moreover, it has been demonstrated that engineered WW domains can bind (non-natural) target ligands with high binding affinities, e.g. in the nM range or higher; whereas to date all reported binding affinities for WW domains are in the μM range. These high binding affinities indicate that engineered WW domains of the invention may have applications in therapeutic and non-therapeutic applications, where binding affinities in the nM or sub-nM range are preferable. Accordingly, the present invention provides therapeutic engineered WW domain peptides and nucleic acids encoding such peptides.

Furthermore, the modified Group IV WW domain peptides of the invention have been demonstrated, for the first time, not only to bind non-natural target ligands; but also to bind non-phosphorylated target ligands that are not bound naturally by the wild-type Group IV Pin1 WW domain sequence. Previously, it was not known that an engineered Pin1 WW domain could be used to recognise non-phosphorylated peptide target sequences. This demonstrates the potential versatility of the WW domain as a novel binding module, which greatly expands the potential repertoire of non-natural target ligands for WW domains.

In addition, it has been shown that a WW domain framework library can be constructed and expressed in order to select modified WW domain peptides for targeting desired ligands. Still more importantly, it has been established that the novel WW domain framework library can be used to alter the specificity of WW domain peptides so that the framework library can be used to select novel binding modules against more than one different non-natural target ligand (such as different peptide sequences). In other words, the framework library is broadly applicable to the selection of new binding modules. A particularly useful screening and selection process is an acellular in vitro selection system, because it offers a larger library size than is currently available with similar cellular based procedures.

Finally, it has been found that modified WW domain peptides can be cyclised while retaining the novel ligand binding activity of the corresponding non-cyclised modified WW domain peptide, and such cyclised WW domain peptides may have particular applications in the field of therapeutics and for other in vivo, in vitro and ex vivo uses.

Further expressions of the inventive concept are set out in the following clauses.

1. A method of making a naïve WW domain peptide library, the method comprising:
(a) providing a plurality of nucleic acids each encoding a WW domain peptide;
(b) introducing diversity into the plurality of nucleic acids, thereby to create a plurality of modified nucleic acids, to provide diversity at one or more amino acid residues in the WW domain peptide of a plurality of said WW domain peptides encoded by the modified nucleic acids; and
(c) expressing the WW domain peptides encoded by said plurality of modified nucleic acids, whereby a library of modified WW domain peptides comprising sequence diversifications is produced.

2. The method of Clause 1, wherein the plurality of nucleic acids in step (a) each encode a WW domain peptide of a defined sequence, and wherein substantially all members of the modified WW domain peptide library in step (c) have at least 50% sequence identity to the defined sequence.

3. The method of Clause 1 or Clause 2, wherein: (i) the naïve WW domain peptide library is derived from a human WW domain, and the plurality of nucleic acids in step (a) encode a human WW domain peptide; and/or (ii) the naïve WW domain peptide library is derived from a Group IV WW domain, and the plurality of nucleic acids in step (a) encode a Group IV WW domain peptide; and/or (iii) the naïve WW domain peptide library is derived from Pin1 WW domain, and the plurality of nucleic acids in step (a) encode a Pin1 WW domain peptide.

4. The method of Clause 3, wherein the Pin1 WW domain peptide sequence comprises the amino acids at positions 6 to 38 of SEQ ID NO: 1 and wherein the naïve WW domain peptide library further comprises diversity at one or more amino acid residues in the Pin1 WW domain peptide sequence.

5. The method of Clause 4, wherein the amino acids at positions 11 and 34 are tryptophan and the amino acid at position 26 is asparagine.

6. The method of Clause 4 or Clause 5, wherein: (i) at least one amino acid is deleted from the sequence of SEQ ID NO: 1 between positions 17 and 20 and/or the methionine at position 15 is changed to tryptophan; (ii) at least one amino acid between positions 17 and 20 (loop 1) is inserted, and/or the methionine at position 15 is changed to tryptophan; (iii) at least one amino acid between positions 17 and 20 (loop 1) is deleted, at least one amino acid between positions 27 and 30 (loop 2) is inserted, and/or the methionine at position 15 is changed to tryptophan; or (iv) at least one amino acid between positions 17 and 20 (loop 1) is inserted, at least one amino acid between positions 27 and 30 (loop 2) is inserted, and/or the methionine at position 15 is changed to tryptophan.

7. The method of any of Clauses 4 to 6, wherein the diversity is introduced at one or more of positions 12, 14, 17, 18, 23, 25, 27, 30 and 32 of SEQ ID NO: 1.

8. The method of any of Clauses 4 to 7, wherein the diversity is introduced at one or more of positions 17, 18 and 32; and/or diversity is introduced at one or more of positions 12, 14, 23, 25, 27 and 30; and/or diversity is introduced at one or more of positions 16, 20, 21, 28 and 29.

9. The method of Clause 8, wherein the diversity is introduced at positions 17, 18 and 32 through the use of the VVM codon, wherein V is A, C or G, and M is A or C; and diversity is introduced at one or more of positions 12, 14, 23, 25, 27 and 30 through the use of the NNB codon, wherein N is A, C, G, or T, and B is C, G or T.

10. The method of any of Clauses 7 to 9, wherein diversity is introduced at 3 or more, 5 or more, 7 or more, or all of positions 12, 14, 17, 18, 23, 25, 27, 30 and 32 of SEQ ID NO: 1.

11. The method of any of Clauses 1 to 10, wherein step (b) further comprises amplifying the nucleic acid sequence, such as using PCR.

12. The method of any of Clauses 1 to 11, which further comprises:
(d) selecting peptides of the library against a target ligand against which the WW domain peptides in step (a) have not been isolated.

13. The method of Clause 12, wherein the selection is performed using an in vitro selection procedure.

14. The method of Clause 12 or Clause 13, wherein the target ligand is not bound by the WW domain peptides in step (a).

15. The method of any of Clauses 12 to 14, wherein the target ligand is an extracellular protein or peptide.

16. The method of any of Clauses 12 to 15, wherein the target ligand is a protein or peptide sequence which is not phosphorylated at serine or threonine amino acids.

17. The method of Clause 16, wherein the target ligand is a non-phosphorylated peptide or protein.

18. The method of any of Clauses 12 to 17, wherein the target ligand is VEGFR2 or NGF.

19. The method of any of Clauses 1 to 18, which further comprises:
(e) isolating peptides that bind to the target ligand with a dissociation constant (Kd) of less than 1 μM, less than 500 nM, or less than 100 nM.

20. A method for isolating a modified WW domain peptide from a naïve WW domain peptide display library, the library comprising a plurality of nucleic acid sequences that encode displayed modified WW domain peptides, comprising the steps of:
(a) expressing a plurality of nucleic acid constructs, wherein each nucleic acid construct comprises a promoter sequence operably linked to the nucleic acid sequence, such that expression of the plurality of nucleic acid constructs results in formation of a plurality of peptide-nucleic acid complexes, each complex comprising at least one displayed modified WW domain peptide associated with the corresponding nucleic acid construct encoding the displayed peptide;
(b) exposing the plurality of peptide-nucleic acid complexes to at least one target ligand, and allowing the peptide-nucleic acid complexes to associate with the ligand, suitably by binding of a displayed modified WW domain peptide to the target ligand;
(c) removing any peptide-nucleic acid complexes that remain unassociated with the target ligand; and
(d) recovering any target ligand-associated peptide-nucleic acid complexes.

21. The method of Clause 20, wherein the naïve WW domain peptide display library is derived from (i) a human WW domain; and/or (ii) a Group IV WW domain; and/or (iii) a Pin1 WW domain.

22. The method of Clause 21, wherein the naïve WW domain peptide display library is derived from a Pin1 WW domain comprising the sequence of amino acids at positions 6 to 38 of SEQ ID NO: 1, wherein at least one amino acid is deleted between positions 17 and 20; and/or the methionine at position 15 is changed to tryptophan; and which further comprises diversity at one or more amino acid residues in the sequence of SEQ ID NO: 1.

23. The method of Clause 21, wherein the naïve WW domain peptide display library is derived from a Pin1 WW domain comprising the sequence of amino acids at positions 6 to 38 of SEQ ID NO: 1, wherein at least one amino acid is inserted between positions 17 and 20; and/or the methionine at position 15 is changed to tryptophan; and which further comprises diversity at one or more amino acid residues in the sequence of SEQ ID NO: 1.

24. The method of Clause 22 or Clause 23, wherein said diversity at one or more amino acid residues in the sequence of SEQ ID NO: 1 comprises changing up to 15 amino acid residues, up to 12 amino acid residues, or up to 10 amino acid residues of the sequence.

25. The method of any of Clauses 21 to 24, wherein one or more of the amino acids at positions 12, 14, 17, 18, 23, 25, 27, 30 and 32 of SEQ ID NO: 1 are changed.

26. The method of Clause 25, wherein one or more of positions 17, 18 and 32; and/or one or more of positions 12, 14, 23, 25, 27 and 30; and/or one or more of positions 16, 20, 21, 28 and 29 are changed.

27. The method of Clause 25 or Clause 26, wherein 3 or more, 5 or more, 7 or more, or all 9 of the amino acids at positions 12, 14, 17, 18, 23, 25, 27, 30 and 32 of SEQ ID NO: 1 are changed.

1. A naïve WW domain peptide library which has a consensus sequence derived from a WW domain peptide sequence which has been diversified by changing the amino acid sequence at one or more positions, and wherein the consensus sequence has at least three invariant tryptophan residues.

2. The naïve WW domain peptide library of claim 1, wherein substantially all functional members have a three-stranded beta-sheet fold.

3. The naïve WW domain peptide library of claim 1, which is derived from a Group IV WW domain peptide sequence.

4. The naïve WW domain peptide library of claim 1, wherein the consensus sequence comprises the amino acid sequence WX₃WX₁₆₋₃₂W, WX₃WX₁₆₋₁₈W, or WX₃WX₁₈₋₃₂W, wherein X is any amino acid.

5. The naïve WW domain peptide library of claim 1, which is derived from the amino acid sequence at positions 6 to 38 of SEQ ID NO: 1.

6. The naïve WW domain peptide library of claim 5, wherein the consensus sequence includes: (i) a tryptophan at positions 11 and 34, and at least one additional tryptophan at one or more of positions 13, 15, 21, 22, 24, 25 and 39; and/or a tryptophan at positions 11 and 34, and asparagine at position 26 of SEQ ID NO: 1.

7. The naïve WW domain peptide library of claim 5, wherein the sequence of SEQ ID NO: 1 comprises at least one mutation selected from: (i) the deletion of at least one amino acid between positions 17 and 20 (loop 1), and the substitution of methionine at position 15 to tryptophan; (ii) the addition of at least one amino acid between positions 17 and 20 (loop 1), and the substitution of methionine at position 15 to tryptophan; (iii) the deletion of at least one amino acid between positions 17 and 20 (loop 1), the addition of at least one amino acid between positions 27 and 30 (loop 2), and the substitution of methionine at position 15 to tryptophan; or (iv) the addition of at least one amino acid between positions 17 and 20 (loop 1), the addition of at least one amino acid between positions 27 and 30 (loop 2), and the substitution of methionine at position 15 to tryptophan.

8. The naïve WW domain peptide library of claim 6, which is further diversified by mutating one or more amino acid of SEQ ID NO: 1 selected from: positions 12, 14, 23, 25, 27 and 30; and/or positions 17, 18 and 32; and/or positions 16, 20, 21, 28 and 29.

9. The naïve WW domain peptide library of claim 5, which comprises the following changes to the sequence of amino acids at positions 6 to 38 of SEQ ID NO: 1: (i) the deletion of an amino acid between positions 17 and 20; (ii) the substitution of methionine at position 15 to tryptophan; and (iii) the mutation of one or more of the amino acids at positions 17, 18 and 32, and/or one or more of the amino acids at positions 12, 14, 23, 25, 27 and 30.

10. The naïve WW domain peptide library of claim 5, wherein the amino acids at positions 12, 14, 23, 25, 27 and 30 are randomly selected from: (i) any naturally occurring or non-natural amino acid; or (ii) any of the 20 naturally occurring amino acids.

11. The naïve WW domain peptide library of claim 5, wherein the amino acids at positions 17, 18 and 32 are randomly selected from any amino acid of the group consisting of A, G, N, K, D, E, R, T, S, P, H and Q.

12. The naïve WW domain peptide library of claim 1, which comprises a sequence selected from:
KLPPGWX₁KX₂WSX₃X₄XₐGRVX₅YX₆NX₇ITX₈AX₉QWERP where X₁ to X₉ represent any amino acid and Xₐ is optionally any amino acid or absent (i.e. SEQ ID NO: 31); or
KLPPGWX₁KX₂WSX₃X₄GRVX₅YX₆NX₇ITX₈AX₉QWERP wherein the amino acids at positions X₁ to X₉ are randomly selected from any amino acid (i.e. SEQ ID NO: 32).

13. The naïve WW domain peptide library of claim 12, wherein the amino acids at positions X₃, X₄ and X₉ are randomly selected from one of the group of amino acids consisting of A, G, N, K, D, E, R, T, S, P, H and Q.

14. A modified WW domain peptide derived from a wild-type WW domain peptide sequence which has been diversified by changing the amino acid sequence at one or more positions, and wherein the modified WW domain peptide binds a target ligand not bound by the wild-type WW domain peptide from which it is derived, provided that no more than 50% of the amino acids of the wild-type sequence are changed.

15. The modified WW domain peptide of claim 14, which comprises the amino acid sequence WX₃WX₁₆₋₃₂W, WX₃WX₁₆₋₁₈W, or WX₃WX₁₈₋₃₂W wherein X is any amino acid.

16. The modified WW domain peptide of claim 14, which is derived from: (i) a Group IV WW domain sequence; (ii) a human WW domain sequence; and/or Pin1 WW domain peptide sequence comprising the amino acids at positions 6 to 38 of SEQ ID NO: 1 and which comprises one or more mutation to the sequence of SEQ ID NO: 1.

17. The modified WW domain peptide of claim 16, wherein the amino acids at positions 11 and 34 are tryptophan and the amino acid at position 26 is asparagine.

18. The modified WW domain peptide of claim 16, wherein: (i) at least one amino acid between positions 17 and 20 is deleted and/or the methionine at position 15 is changed to tryptophan; (ii) at least one amino acid between positions 17 and 20 (loop 1) is inserted, and/or the methionine at position 15 is changed to tryptophan; (iii) at least one amino acid between positions 17 and 20 (loop 1) is deleted, at least one amino acid between positions 27 and 30 (loop 2) is inserted, and/or the methionine at position 15 is changed to tryptophan; or (iv) at least one amino acid between positions 17 and 20 (loop 1) is inserted, at least one amino acid between positions 27 and 30 (loop 2) is inserted, and/or the methionine at position 15 is changed to tryptophan.

19. The modified WW domain peptide of any of claim 16, which comprises a mutation at one or more amino acid of SEQ ID NO: 1 selected from: positions 12, 14, 23, 25, 27 and 30; and/or positions 17, 18 and 32; and/or positions 16, 20, 21, 28 and 29.

20. The modified WW domain peptide of claim 16, which comprises mutations at 3 or more, 5 or more, 7 or more, or 9 amino acids of SEQ ID NO: 1 selected from positions 12, 14, 17, 18, 23, 25, 27, 30 and 32.

21. The modified WW domain peptide of claim 16, wherein the amino acids at positions 12, 14, 23, 25, 27 and 30 are selected from any one of the 20 naturally occurring amino acids, and wherein the amino acids at positions 17, 18 and 32 are selected from any one of the amino acid of the group consisting of A, G, N, K, D, E, R, T, S, P, H and Q.

22. The modified WW domain peptide of claim 14, which binds: (i) a non-phosphorylated target ligand; (ii) a peptide or protein target ligand; (iii) an extracellular target ligand; and/or (iv) the target ligand with a dissociation constant (Kd) of less than 1 μM, less than 500 nM, less than 200 nM, or less than 100 nM.

23. The modified WW domain peptide of claim 14, wherein the target ligand is VEGFR2 or β-NGF.

24. The modified WW domain peptide of claim 14, which comprises the amino acid sequence at positions 6 to 38 of any one of SEQ ID NOs: 15 to 20 or 26 to 29, based on the numbering of SEQ ID NO: 1.

25. (canceled)

26. (canceled)

27. The modified WW domain peptide of claim 14, which is conjugated to a non-WW domain moiety is selected from an antibody or antibody fragment, or a DNA-binding domain.

28. The modified WW domain peptide of claim 14, which is a cyclic peptide; optionally wherein the peptide has a covalent bond or linkage between the amino acid at position 6 and the amino acid at position 38, or between the amino acid at position 4 and the amino acid at position 29 of SEQ ID NO: 1.

29. A method for antagonising or agonising the function of an extracellular target ligand using the modified WW domain peptide of claim 14.

30-33. (canceled)

34. A method of treating, preventing or alleviating a disease selected from cancer, degenerative disease of the retina, or pain, comprising administering to a subject in need thereof a therapeutically effective amount of the modified WW domain peptide of claim 14.

35-43. (canceled)

44. The naïve WW domain peptide library of claim 1, which is derived from a Group IV WW domain peptide sequence, wherein the Group IV WW domain is Pin1.
